(19) United States
(12) Patent Application Publication
(10) Pub. No.: US 2003/0059792 A1
(43) Pub. Date: Mar. 27, 2003

(54) Models and methods for determining systemic properties of regulated reaction networks

(76) Inventors: Bernhard O. Palsson, La Jolla, CA (US); Markus W. Covert, San Diego, CA (US); Christophe H. Schilling, San Diego, CA (US)

Correspondence Address: GARY CARY WARE & FRIENDENRICH LLP, 4365 EXECUTIVE DRIVE, SUITE 1600, SAN DIEGO, CA, 92121-2189, US

(21) Appl. No.: 10/087,441

(22) Filed: Mar. 1, 2002

Related U.S. Application Data
(60) Provisional application No. 60/272,754, filed on Mar. 1, 2001.
(60) Provisional application No. 60/323,028, filed on Sep. 14, 2001.

(51) Int. Cl. C12Q 1/68; G01N 33/53; G06G 7/48; G06G 7/58; G06F 19/00

(52) U.S. Cl. 435/6; 435/7.1; 702/19; 702/20; 703/11

(57) ABSTRACT

The invention provides a model of a reaction network integrated with regulatory controls related to the reactions. A method is provided for determining a systemic property of a reaction network using a model of the invention. Also provided is a method for modeling changes that occur in a reaction network at various time points due to regulatory events.

This application is based on and claims benefit of U.S. Provisional Application No. 60/272,754, filed Mar. 1, 2001, and U.S. Provisional Application No. 60/323,028, filed Sep. 14, 2001, both of which are incorporated herein by reference.

### BACKGROUND OF THE INVENTION

This invention was made with United States Government support under grant number BES-9814092 awarded by the National Science Foundation of the United States. The U.S. Government may have certain rights in this invention.

This invention relates generally to computational approaches for the analysis of biological systems and, more specifically, to computer readable media and methods for simulating and predicting the activity of regulated biological reaction networks.

All cellular behaviors involve the simultaneous function and integration of many interrelated genes, gene products and chemical reactions. Because of this interconnectivity, it is virtually impossible to a priori predict the effect of a change in a single gene or gene product, or the effect of a drug or an environmental factor, on cellular behavior. The ability to accurately predict cellular behavior under different conditions would be extremely valuable in many areas of medicine and industry. For example, if it were possible to predict which gene products are suitable drug targets, it would considerably shorten the time it takes to develop an effective antibiotic or anti-tumor agent. Likewise, if it were possible to predict the optimal fermentation conditions and genetic make-up of a microorganism for production of a particular industrially important product, it would allow for rapid and cost-effective improvements in the performance of these microorganisms.

Computational approaches have recently been developed to reconstruct biological reaction networks that occur within organisms, with the goal of being able to predict and analyze organismal behavior. One of the most powerful current approaches involves constraints-based modeling, which provides a mathematically defined “solution space” wherein all possible behaviors of the biological system must lie. The solution space can then be explored to determine the range of capabilities and preferred behavior of the biological system under various conditions. Models that utilize reaction networks derived in large part from genome sequence data have been developed for a number of organisms, and are referred to as “genome-scale” models.

In current constraints-based models, all reactions in the network are considered to always be available unless a decision is made by the individual modeler to remove the reaction, such as when simulating the effect of a gene deletion. This implies that all of the required proteins for all reactions are functionally present in the system and that their associated genes are always expressed. Additionally, in current constraints-based models, a reaction is allowed to occur so long as the necessary substrates are available. However, in nature this is not the case, because complex regulatory controls are placed on biological systems that allow certain reactions to only occur under particular conditions.

Whether a reaction actually occurs in an organism is dependent on a large number of regulatory factors and events apart from just the presence of the necessary substrates. These regulatory factors and events can regulate the activity of proteins or enzymes involved in the reaction, regulate cofactors that stabilize or destabilize protein or enzyme structure, regulate the assembly of proteins or enzymes, regulate the translation of mRNA into proteins, regulate the transcription of genes into mRNA, assist in controlling any of these processes, or act by mechanisms that are as yet unknown.

Current constraints-based models that attempt to describe cellular behavior do not take into account these complex regulatory controls that determine whether particular reactions in the network actually occur. Therefore, current models cannot accurately predict or describe the effect of environmental or genetic changes. Thus, there exists a need for models and modeling methods that can be used to accurately simulate and effectively analyze the behavior of organisms under a variety of conditions. The present invention satisfies this need and provides related advantages as well.

### BRIEF DESCRIPTION OF THE DRAWINGS

FIG. 1 shows a flow diagram illustrating a method for developing and implementing a regulated biochemical reaction network model.

FIG. 2 shows, in Panel A, an exemplary biochemical reaction network; in Panel B, an exemplary regulatory control structure for the reaction network in panel A; in Panel C, an exemplary simulated flux distribution for the biochemical reaction network in Panel A without regulatory constraints considered; and in Panel D a simulated flux distribution for a biochemical reaction network in which the regulatory constraints depicted in Panel B are included.

FIG. 3 shows a schematic drawing of a regulatory network associated with a reaction in a metabolic network.

FIG. 4 shows a schematic drawing of a reaction that is acted upon by a regulatory event.

FIG. 5 shows a flow diagram illustrating a transient or time-dependent implementation of a regulated biochemical reaction network model.

FIG. 6 shows a flow diagram illustrating a method for developing a genome scale regulated model of a biochemical reaction network.

FIG. 7 shows a schematic drawing of a simplified core metabolic network, together with a table containing the stoichiometry of the 20 metabolic reactions included in the network.

FIG. 8 shows, in Panel A, a simulation of aerobic growth of E. coli on acetate with glucose reutilization; in Panel B, a table of parameters used to generate the plots in Panel A; and in Panel C, in silico arrays showing the up- or down-regulation of selected genes, or activity of regulatory proteins, in the regulatory network.

FIG. 9 shows, in Panel A, a simulation of anaerobic growth of E. coli on glucose; in Panel B, a table of parameters used to generate the plots in Panel A; and in Panel C, in silico arrays showing the up- or down-regulation of selected genes, or activity of regulatory proteins, in the regulatory network.

FIG. 10 shows, in Panel A, a simulation of aerobic growth of E. coli on glucose and lactose; in Panel B, a table of parameters used to generate the plots in Panel A; and in Panel C, in silico arrays showing the up- or down-regulation of selected genes, or activity of regulatory proteins, in the regulatory network.

### SUMMARY OF THE INVENTION

The invention provides a computer readable medium or media, including (a) a data structure relating a plurality of reactants to a plurality of reactions of a biochemical reaction network, wherein each of the reactions includes a reactant identified as a substrate of the reaction, a reactant identified as a product of the reaction and a stoichiometric coefficient relating the substrate and the product, and wherein at least one of the reactions is a regulated reaction; and (b) a constraint set for the plurality of reactions, wherein the constraint set includes a variable constraint for the regulated reaction.

The invention further provides a method for determining a systemic property of a biochemical reaction network. The method includes the steps of (a) providing a data structure relating a plurality of reactants to a plurality of reactions of a biochemical reaction network, wherein each of the reactions includes a reactant identified as a substrate of the reaction, a reactant identified as a product of the reaction and a stoichiometric coefficient relating the substrate and the product, and wherein at least one of the reactions is a regulated reaction; (b) providing a constraint set for the plurality of reactions, wherein the constraint set includes a variable constraint for the regulated reaction; (c) providing a condition-dependent value to the variable constraint; (d) providing an objective function, and (e) determining at least one flux distribution that minimizes or maximizes the objective function when the constraint set is applied to the data structure, thereby determining a systemic property of the biochemical reaction network.

Also provided by the invention is a method for determining a systemic property of a biochemical reaction network at a first and second time. The method includes the steps of (a) providing a data structure relating a plurality of reactants to a plurality of reactions of a biochemical reaction network, wherein each of the reactions includes a reactant identified as a substrate of the reaction, a reactant identified as a product of the reaction and a stoichiometric coefficient relating the substrate and the product, and wherein at least one of the reactions is a regulated reaction; (b) providing a constraint set for the plurality of reactions, wherein the constraint set includes a variable constraint for the regulated reaction; (c) providing a condition-dependent value to the variable constraint; (d) providing an objective function; (e) determining at least one flux distribution at a first time that minimizes or maximizes the objective function when the constraint set is applied to the data structure, thereby determining a systemic property of the biochemical reaction network at the first time; (f) modifying the value provided to the variable constraint, and (g) repeating step (e), thereby determining a systemic property of the biochemical reaction network at a second time.

### DETAILED DESCRIPTION OF THE INVENTION

The present invention provides an in silico model of a regulated reaction network such as a biochemical reaction network found in a biological system. The model of the invention defines a range of allowed activities for the reaction network as a whole, thereby defining a solution space that contains any and all possible functionalities of the reaction network. According to the invention, regulatory events can be incorporated into the model by utilizing a function that represents the activity or outcome of a regulatory event. An advantage of accounting for regulatory events that occur in the reaction network is that, because regulation reduces the range of activities for a reaction network, the solution space can be made smaller, thereby increasing the predictive capabilities of the in silico models.

A solution space is defined by constraints such as the well-known stoichiometry of metabolic reactions as well as reaction thermodynamics and capacity constraints associated with maximum fluxes through reactions. These are examples of physical-chemical constraints that all systems must abide by. Using the models and methods of the invention, the space defined by these constraints can be explored to determine the phenotypic capabilities and preferred behavior of the biological system using analysis techniques such as convex analysis, linear programming and the calculation of extreme pathways as described, for example, in Schilling et al., J. Theor. Biol. 203:229-248 (2000); Schilling et al., Biotech. Bioeng. 71:286-306 (2000) and Schilling et al., Biotech. Prog. 15:288-295 (1999). As such, the space contains any and all possible functionalities of the reconstituted network.

For a reaction network that is defined for a complete organism through the use of genome sequence, biochemical, and physiological data this solution space describes the functional capabilities of the organism as described for example in WO 00/46405. This general approach to developing cellular models is known in the art as constraints-based modeling and includes methods such as flux balance analysis, metabolic pathway analysis, and extreme pathway analysis. Genome scale models have been created for a number of organisms including Escherichia coli (Edwards et al., Proc. Natl. Acad. Sci. USA 97:5528-5533 (2000)), Haemophilus influenzae (Edwards et al., J. Biol. Chem. 274: 17410-17416 (1999)), and Helicobacter pylori. These and other constraints-based models known in the art can be modified according to the methods of the present invention in order to produce models capable of predicting the effects of regulation on systemic properties or to predict holistic functions of these organisms.

Once the solution space has been defined, it can be analyzed to determine possible solutions under various conditions. One approach is based on metabolic flux balancing in a metabolic steady state which can be performed as described in Varma and Palsson, Biotech. 12:994-998 (1994). Flux balance approaches can be applied to metabolic networks to simulate or predict systemic properties of adipocyte metabolism as described in Fell and Small, J. Biochem. 138:781-786 (1986), acetate secretion from E. coli under ATP maximization conditions as described in Majewski and Domach, Biotech. Bioeng. 35:732-738 (1990) and ethanol secretion by yeast as described in Vanrolleghem et al. Biotech. Prog. 12:434-448 (1996). Additionally, this approach can be used to predict or simulate the growth of E. coli on a variety of single-carbon sources as well as the metabolism of H. influenzae as described in Edwards and Palsson, Proc. Natl. Acad. Sci. 97:5528-5533 (2000), Edwards and Palsson, J. Bio. Chem. 274:17410-17416 (1999) and Edwards et al., Nature Biotech. 19:125-130 (2001).

As useful as the defined solution spaces resulting from stand-alone constraints-based models are for conceptual and basic scientific purposes, they have limited predictive ability, due to their large volume and dimensionality. The present invention provides methods to incorporate constraints that are associated with how the functional operation of reaction networks are controlled/regulated. An advantage of the invention is that the dimensionality and volume of the solution spaces can be reduced due to the incorporation of regulatory constraints into a constraints-based model, thereby improving the predictive capabilities of the model. Accordingly, the range of possible phenotypes that result for a particular mutation or set of mutations can be more readily predicted by incorporating the regulatory constraints of the, invention into a constraints-based model.

The invention provides a computer readable medium or media, including (a) a data structure relating a plurality of reactants to a plurality of reactions of a biochemical reaction network, wherein each of the reactions includes a reactant identified as a substrate of the reaction, a reactant identified as a product of the reaction and a stoichiometric coefficient relating the substrate and the product, and wherein at least one of the reactions is a regulated reaction; and (b) a constraint set for the plurality of reactions, wherein the constraint set includes a variable constraint for the regulated reaction.

As used herein, the term “biochemical reaction network” is intended to mean a collection of chemical conversions that are capable of occurring in or by a viable biological organism. Chemical conversions that are capable of occurring in or by a viable biological organism can include, for example, reactions that naturally occur in a particular organism such as those referred to below; reactions that naturally occur in a subset of organisms, such as those in a particular kingdom, phylum, genera, family, species or environmental niche; or reactions that are ubiquitous in nature. Chemical conversions that are capable of occurring in or by a viable biological organism can include, for example, those that occur in eukaryotic cells, prokaryotic cells, single celled organisms or multicellular organisms. A collection of chemical conversions included in the term can be substantially complete or can be a subset of reactions including, for example, reactions involved in metabolism such as central or peripheral metabolic pathways, reactions involved in signal transduction, reactions involved in growth or development, or reactions involved in cell cycle control.

Central metabolic pathways include the reactions that belong to glycolysis, pentose phosphate pathway (PPP), tricarboxylic acid (TCA) cycle and respiration.

A peripheral metabolic pathway is a metabolic pathway that includes one or more reactions that are not a part of a central metabolic pathway. Examples of reactions of peripheral metabolic pathways that can be represented in a data structure or model of the invention include those that participate in biosynthesis of an amino acid, degradation of an amino acid, biosynthesis of a purine, biosynthesis of a pyrimidine, biosynthesis of a lipid, metabolism of a fatty acid, biosynthesis of a cofactor, metabolism of a cell wall component, transport of a metabolite or metabolism of a carbon source, nitrogen source, phosphate source, oxygen source, sulfur source or hydrogen source.

As used herein, the term “reaction” is intended to mean a chemical conversion that consumes a substrate or forms a product. A chemical conversion included in the term can occur due to the activity of one or more enzymes that are genetically encoded by an organism or can occur spontaneously in a cell or organism. A chemical conversion included in the term includes, for example, a conversion of a substrate to a product such as one due to nucleophilic or electrophilic addition, nucleophilic or electrophilic substitution, elimination, reduction or oxidation or changes in location such as those that occur when a reactant is transported across a membrane or from one compartment to another. The substrate and product of a reaction can be differentiated according to location in a particular compartment even though they are chemically the same. Thus, a reaction that transports a chemically unchanged reactant from a first compartment to a second compartment has as its substrate the reactant in the first compartment and as its product the reactant in the second compartment. The term can include a conversion that changes a macromolecule from a first conformation, or substrate conformation, to a second conformation, or product conformation. Such conformational changes can be due, for example, to transduction of energy due to binding a ligand such as a hormone or receptor, or from a physical stimulus such as absorption of light. It will be understood that when used in reference to an in silico model or data structure a reaction is intended to be a representation of a chemical conversion that consumes a substrate or produces a product.

As used herein, the term “regulated,” when used in reference to a reaction in a data structure, is intended to mean a reaction that experiences an altered flux due to a change in the value of a constraint or a reaction that has a variable constraint.

As used herein, the term “regulatory reaction” is intended to mean a chemical conversion or interaction that alters the activity of a catalyst. A chemical conversion or interaction can directly alter the activity of a catalyst such as occurs when a catalyst is post-translationally modified or can indirectly alter the activity of a catalyst such as occurs when a chemical conversion or binding event leads to altered expression of the catalyst. Thus, transcriptional or translational regulatory pathways can indirectly alter a catalyst or an associated reaction. Similarly, indirect regulatory reactions can include reactions that occur due to downstream components or participants in a regulatory reaction network. When used in reference to a data structure or in silico model, the term is intended to mean a first reaction that is related to a second reaction by a function that alters the flux through the second reaction by changing the value of a constraint on the second reaction.

As used herein, the term “reactant” is intended to mean a chemical that is a substrate or a product of a reaction. The term can include substrates or products of reactions catalyzed by one or more enzymes encoded by an organism's genome, reactions occurring in an organism that are catalyzed by one or more non-genetically encoded catalysts, or reactions that occur spontaneously in a cell or organism. Metabolites are understood to be reactants within the meaning of the term. It will be understood that when used in the context of an in silico model or data structure, a reactant is understood to be a representation of chemical that is a substrate or product of a reaction.

As used herein the term “substrate” is intended to mean a reactant that can be converted to one or more products by a reaction. The term can include, for example, a reactant that is to be chemically changed due to nucleophilic or electrophilic addition, nucleophilic or electrophilic substitution, elimination, reduction or oxidation or that is to change location such as by being transported across a membrane or to a different compartment. The term can include a macromolecule that changes conformation due to transduction of energy.

As used herein, the term “product” is intended to mean a reactant that results from a reaction with one or more substrates. The term can include, for example, a reactant that has been chemically changed due to nucleophilic or electrophilic addition, nucleophilic or electrophilic substitution, elimination, reduction or oxidation or that has changed location such as by being transported across a membrane or to a different compartment. The term can include a macromolecule that changes conformation due to transduction of energy.

As used herein, the term “data structure” is intended to mean a representation of information in a format that can be manipulated or analyzed. A format included in the term can be, for example, a list of information, a matrix that correlates two or more lists of information, a set of equations such as linear algebraic equations, or a set of Boolean statements. Information included in the term can be, for example, a substrate or product of a chemical reaction, a chemical reaction relating one or more substrates to one or more products, or a constraint placed on a reaction. Thus, a data structure of the invention can be a representation of a reaction network such as a biochemical reaction network.

A plurality of reactants can be related to a plurality of reactions in any data structure that represents for each reactant, the reactions by which it is consumed or produced. Thus, the data structure serves as a representation of a biological reaction network or system. A reactant in a plurality of reactants or a reaction in a plurality of reactions that are included in a data structure of the invention can be annotated. Such annotation can allow each reactant to be identified according to the chemical species and the cellular compartment in which it is present. Thus, for example, a distinction can be made between glucose in the extracellular compartment versus glucose in the cytosol. A data structure can include a first substrate or product in the plurality of reactions that is assigned to a first compartment and a second substrate or product in the plurality of reactions that is assigned to a second compartment. Examples of compartments to which reactants can be assigned include the intracellular space of a cell; the extracellular space around a cell; the interior space of an organelle such as a mitochondrium, endoplasmic reticulum, golgi apparatus, vacuole or nucleus; or any subcellular space that is separated from another by a membrane. Additionally each of the reactants can be specified as a primary or secondary metabolite. Although identification of a reactant as a primary or secondary metabolite does not indicate any chemical distinction between the reactants in a reaction, such a designation can assist in visual representations of large networks of reactions.

The reactants to be used in a data structure or model of the invention can be obtained from or stored in a compound database. Such a compound database can be a universal data base that includes compounds from a variety of organisms or, alternatively, can be specific to a particular organism or reaction network. The reactions included in a data structure or model of the invention can be obtained from a metabolic reaction database that includes the substrates, products, and stoichiometry of a plurality of metabolic reactions of a particular organism.

A reaction that is represented in a data structure or model of the invention can be annotated to indicate a macromolecule that catalyzes the reaction or an open reading frame that expresses the macromolecule. Other annotation information can include, for example, the name(s) of the enzyme(s) catalyzing a particular reaction, the gene(s) that code for the enzymes, the EC number of the particular metabolic reaction, a subset of reactions to which the reaction belongs, citations to references from which information was obtained, or a level of confidence with which a reaction is believed to occur in a particular biochemical reaction network or organism. Such information can be obtained during the course of building a metabolic reaction database or model of the invention as described below. Annotated reactions that are used in a data structure or model of the invention can be obtained from or stored in a gene database that relates one or more reactions with one or more genes or proteins in a particular organism.

As used herein, the term “stoichiometric coefficient” is intended to mean a numerical constant correlating the quantity of one or more reactants and one or more products in a chemical reaction. The reactants in a data structure or model of the invention can be designated as either substrates or products of a particular reaction, each with a discrete stoichiometric coefficient assigned to them to describe the chemical conversion taking place in the reaction. Each reaction is also described as occurring in either a reversible or irreversible direction. Reversible reactions can either be represented as one reaction that operates in both the forward and reverse direction or be decomposed into two irreversible reactions, one corresponding to the forward reaction and the other corresponding to the backward reaction.

The systems and methods described herein can be implemented on any conventional host computer system, such as those based on Intel.RTM. microprocessors and running Microsoft Windows operating systems. Other systems, such as those using the UNIX or LINUX operating system and based on IBM.RTM., DEC.RTM. or Motorola.RTM. microprocessors are also contemplated. The systems and methods described herein can also be implemented to run on client-server systems and wide-area networks, such as the Internet.

Software to implement a method or system of the invention can be written in any well-known computer language, such as Java, C, C++, Visual Basic, FORTRAN or COBOL and compiled using any well-known compatible compiler. The software of the invention normally runs from instructions stored in a memory on a host computer system. A memory or computer readable medium can be a hard disk, floppy disc, compact disc, magneto-optical disc, Random Access Memory, Read Only Memory or Flash Memory. The memory or computer readable medium used in the invention can be contained within a single computer or distributed in a network, A network can be any of a number of conventional network systems known in the art such as a local area network (LAN) or a wide area network (WAN). Client-server environments, database servers and networks that can be used in the invention are well known in the art. For example, the database server can run on an operating system such as UNIX, running a relational database management system, a World Wide Web application and a World Wide Web server. Other types of memories and computer readable media are also contemplated to function within the scope of the invention.

The invention further provides a method for determining a systemic property of a biochemical reaction network. The method includes the steps of (a) providing a data structure relating a plurality of reactants to a plurality of reactions of a biochemical reaction network, wherein each of the reactions includes a reactant identified as a substrate of the reaction, a reactant identified as a product of the reaction and a stoichiometric coefficient relating the substrate and the product, and wherein at least one of the reactions is a regulated reaction; (b) providing a constraint set for the plurality of reactions, wherein the constraint set includes a variable constraint for the regulated reaction; (c) providing a condition-dependent value to the variable constraint; (d) providing an objective function, and (e) determining at least one flux distribution that minimizes or maximizes the objective function when the constraint set is applied to the data structure, thereby determining a systemic property of the biochemical reaction network.

As used herein, the term “systemic property” is intended to mean a capability or quality of an organism as a whole. The term can also refer to a dynamic property which is intended to be the magnitude or rate of a change from an initial state of an organism to a final state of the organism. The term can include the amount of a chemical consumed or produced by an organism, the rate at which a chemical is consumed or produced by an organism, the amount or rate of growth of an organism or the amount of or rate at which energy, mass or electron flow through a particular subset of reactions of an organism.

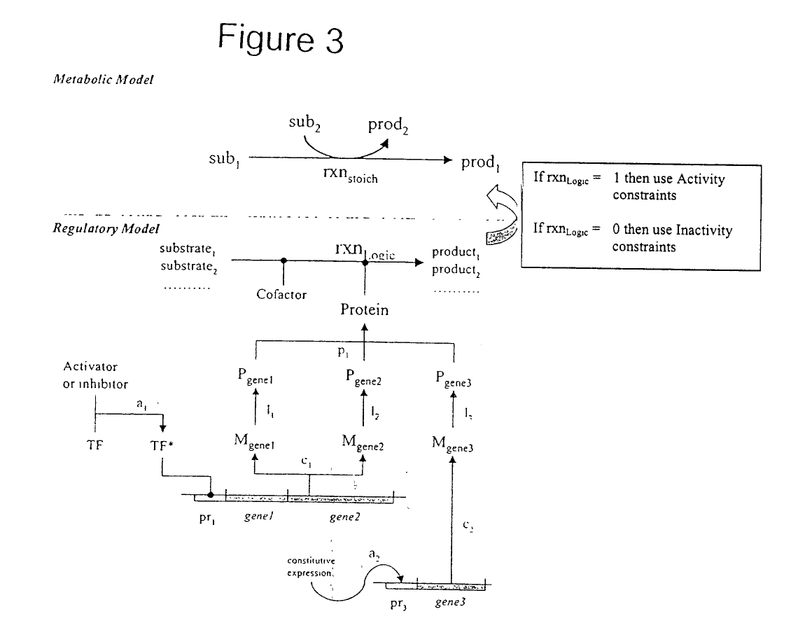

As used herein, the term “regulatory data structure” is intended to mean a representation of an event, reaction or network of reactions that activate or inhibit a reaction, the representation being in a format that can be manipulated or analyzed. An event that activates a reaction can be an event that initiates the reaction or an event that increases the rate or level of activity for the reaction. An event that inhibits a reaction can be an event that stops the reaction or an event that decreases the rate or level of activity for the reaction. Reactions that can be represented in a regulatory data structure include, for example, reactions that control expression of a macromolecule that catalyzes a reaction such as transcription and translation reactions, reactions that lead to post translational modification of a protein or enzyme such as phophorylation, dephosphorylation, prenylation, methylation, oxidation or covalent modification, reaction that process a protein or enzyme such as removal of a pre or pro sequence, reactions that degrade a protein or enzyme or reactions that lead to assembly of a protein or enzyme. An example of a network of reactions that can be represented by a regulatory data structure are shown in FIG. 3.

As used herein, the term “regulatory event” is intended to mean a modifier of the flux through a reaction that is independent of the amount of reactants available to the reaction. A modification included in the term can be a change in the presence, absence, or amount of an enzyme that catalyzes a reaction. A modifier included in the term can be a regulatory reaction such as a signal transduction reaction or an environmental condition such as a change in pH, temperature, redox potential or time. It will be understood that when used in reference to an in silico model or data structure a regulatory event is intended to be a representation of a modifier of the flux through a reaction that is independent of the amount of reactants available to the reaction.

As used herein, the term “constraint” is intended to mean an upper or lower boundary for a reaction. A boundary can specify a minimum or maximum flow of mass, electrons or energy through a reaction. A boundary can further specify directionality of a reaction. A boundary can be a constant value such as zero, infinity, or a numerical value such as an integer. Alternatively, a boundary can be a variable boundary value as set forth below.

As used herein, the term “variable,” when used in reference to a constraint is intended to mean capable of assuming any of a set of values in response to being acted upon by a function. The term “function” is intended to be consistent with the meaning of the term as it is understood in the computer and mathematical arts. A function can be binary such that changes correspond to a reaction being off or on. Alternatively, continuous functions can be used such that changes in boundary values correspond to increases or decreases in activity. Such increases or decreases can also be binned or effectively digitized by a function capable of converting sets of values to discreet integer values. A function included in the term can correlate a boundary value with the presence, absence or amount of a biochemical reaction network participant such as a reactant, reaction, enzyme or gene. A function included in the term can correlate a boundary value with an outcome of at least one reaction in a reaction network that includes the reaction that is constrained by the boundary limit. A function included in the term can also correlate a boundary value with an environmental condition such as time, pH, temperature or redox potential.

The ability of a reaction to be actively occurring is dependent on a large number of additional factors beyond just the availability of substrates. These factors, which can be represented as variable constraints in the models and methods of the invention include, for example, the presence of cofactors necessary to stabilize the protein/enzyme, the presence or absence of enzymatic inhibition and activation factors, the active formation of the protein/enzyme through translation of the corresponding mRNA transcript, the transcription of the associated gene(s), the presence of chemical signals and/or proteins that assist in controlling these processes that ultimately determine whether a chemical reaction is capable of being carried out within an organism.

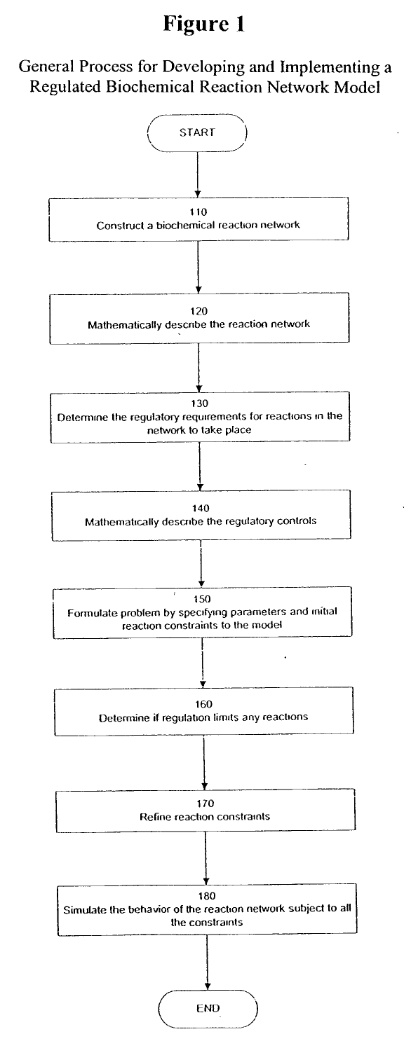

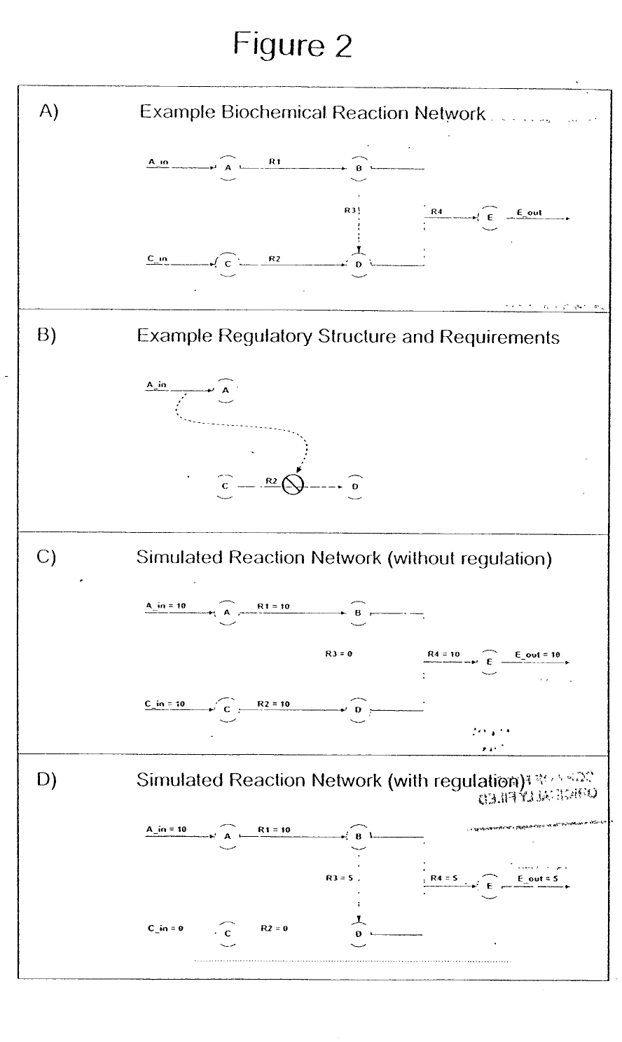

FIG. 1 shows a general process 100 for the development and implementation of a regulated model of a biochemical reaction network. The process starts with step 110 wherein a data structure representing a biochemical reaction network is constructed. The process can start with the generation of a reaction index listing all of the reactions which can occur in the network along with the net reaction equations. As set forth above, such a list can be derived from or stored in a reaction database. If the example reaction network depicted in FIG. 2A is considered, there are 4 balanced biochemical reactions interconverting 5 metabolites. There are 3 exchange reactions that are added to enable the input and output of certain metabolites. The reaction index for this network contains 7 reactions and is as follows:

| | | |
|---|---|---|
| 1. | R1: | A → B |
| 2. | R2: | C → D |
| 3. | R3: | B → D |
| 4. | R4: | B + D → E |
| 5. | A_in: → A | |
| 6. | C_in: → C | |
| 7. | E_out: | E → |

Reactions included in a model of the invention can include intra-system or exchange reactions. Intra-system reactions are the chemically and electrically balanced interconversions of chemical species and transport processes, which serve to replenish or drain the relative amounts of certain metabolites. These intra-system reactions can be classified as either being transformations or translocations. A transformation is a reaction that contains distinct sets of compounds as substrates and products, while a translocation contains reactants located in different compartments. Thus, a reaction that simply transports a metabolite from the extracellular environment to the cytosol, without changing its chemical composition is solely classified as a translocation, while a reaction such as the phosphotransferase system (PTS) which takes extracellular glucose and converts it into cytosolic glucose-6-phosphate is a translocation and a transformation.

Exchange reactions are those which constitute sources and sinks, allowing the passage of metabolites into and out of a compartment or across a hypothetical system boundary. These reactions are included in a model for simulation purposes and represent the metabolic demands placed on a particular organism. While they may be chemically balanced in certain cases, they are typically not balanced and can often have only a single substrate or product. As a matter of convention the exchange reactions are further classified into demand exchange and input/output exchange reactions.

Input/output exchange reactions are used to allow extracellular reactants to enter or exit the reaction network/system. For each of the extracellular metabolites a corresponding input/output exchange reaction can be created. These reactions are always reversible with the metabolite indicated as a substrate with a stoichiometric coefficient of one and no products produced by the reaction. This particular convention is adopted to allow the reaction to take on a positive flux value (activity level) when the metabolite is being produced or drained out of the system and a negative flux value when the metabolite is being consumed or introduced into the system. These reactions will be further constrained during the course of a simulation to specify exactly which metabolites are available to the cell and which can be excreted by the cell.

A demand exchange reaction is always specified as an irreversible reaction containing at least one substrate. These reactions are typically formulated to represent the production of an intracellular metabolite by the metabolic network or the aggregate production of many reactants in balanced ratios such as in the representation of a growth reaction. The demand exchange reactions can be introduced for any metabolite in the model. Most commonly these reactions are introduced on metabolites that are required to be produced by the cell for the purposes of creating a new cell such as amino acids, nucleotides, phospholipids, and other biomass constituents, or metabolites that are to be produced for alternative purposes. Once these metabolites are identified, a demand exchange reaction that is irreversible and specifies the metabolite as a substrate with a stoichiometric coefficient of one can be created. With these specifications, if the reaction is active it leads to the net production of the metabolite by the system meeting potential production demands. Examples of processes that can be represented in a reaction network data structure and analyzed by the methods of the invention include, for example, protein expression levels and growth rate.

In addition to these demand exchange reactions that are placed on individual metabolites, demand exchange reactions that utilize multiple metabolites in defined stoichiometric ratios can be introduced. These reactions are referred to as aggregate demand exchange reactions. Like all exchange reactions they are balanced chemically. An example of an aggregate demand reaction would be a reaction used to simulate the concurrent growth demands or production requirements associated with cell growth that are placed on a cell.

The process then moves on to step 120 in which a mathematical representation of the network is generated from this list of reactions to create a data structure. This is accomplished using known practices in the art leading to a list of dynamic mass balance equations for each of the metabolites describing the change in concentration of the metabolite over time as the difference between the rates of production and the rates of consumption of the metabolites by the various reactions in which it participates as a substrate or product (see, for example, Schilling et al., J. Theor. Biol. 203:229-248 (2000)). When considering a pseudo steady state these dynamic mass balances convert into a series of linear equations describing the balancing of metabolites in the network. For the example network in FIG. 2A., the linear mass balance equations are as follows:

0=A_{—}in−R1

0=R1−R3−R4

0=C_{—}in−R2

0=R2+R3−R4

0=R4−E_{—}out

Due to thermodynamic principles, chemical reactions can effectively be either reversible or irreversible in nature. This leads to the imposition of constraints on the directional flow of the flux through a reaction. If a reaction is deemed irreversible then the flux is constrained to be positive, and if it is reversible it can take on any value positive or negative. For the example network, the reactions are all considered to be irreversible leading to the following set of constraints expressed as a series of linear inequalities:

0≦R1≦∞

0≦R2≦∞

0≦R3≦∞

0≦R4≦∞

0≦A_{—}in≦∞

0≦C_{—}in≦∞

0≦E_{—}out≦∞

Collectively these 5 linear equations and 7 linear inequalities describe the reaction network under steady state conditions and represent the constraints placed on the network by stoichiometry and reaction thermodynamics.

The process 100 then continues to step 130 wherein any known regulation of the reactions in the biochemical reaction network is determined. This leads to the construction of a regulatory network which interacts with the reaction network. For the example network in FIG. 2, reaction R2 is the only reaction that is regulated. It is controlled in a manner whereby if metabolite A is present and available for uptake by the network the reaction R2 is inhibited from proceeding. This will prevent metabolite C from being used by the network. This is analogous to the concept of catabolite repression that is commonly seen in prokaryotes such as E. coli and is illustrated in further detail in the Examples below. This basic regulatory reaction is illustrated in FIG. 2B.

With the regulation of reactions determined, the process 100 moves to step 140 wherein the regulatory network is described mathematically and used to create a regulatory data structure. A regulatory data structure can represent regulatory reactions as Boolean logic statements. For each reaction in the network a Boolean variable can be introduced (Reg-reaction). The variable takes on a value of 1 when the reaction is available for use in the reaction network and will take on a value of 0 if the reaction is restrained due to some regulatory feature. A series of Boolean statements can then be introduced to mathematically represent the regulatory network. For the example network the regulatory data structure is described as follows:

Reg-R1=1

Reg-R2=IF NOT(A_in)

Reg-R3=1

Reg-R4=1

Reg-A_in=1

Reg-C_in=1

Reg-E_out=1

These statements indicate that R2 can occur if reaction A_in is not occurring (i.e. if metabolite A is not present). Similarly, it is possible to assign the regulation to a variable A which would indicate the presence or absence of A above or below a threshold concentration that leads to the control of R2. This form of representing the regulation is described in the Examples below. Any function that provides values for variables corresponding to each of the reactions in the biochemical reaction network whose values will indicate if the reaction can proceed according to the regulatory structure can be used in to represent a regulatory reaction or set of regulatory reactions in a regulatory data structure.

The combined linear equations and inequalities of step 120 and the Boolean statements generated in step 140 represent an integrated model of the biochemical reaction network and its regulation. Such a model for a metabolic reaction network is provided in the Examples and is referred to as a combined metabolic/regulatory reaction model. An integrated model of the invention can then be implemented to perform simulations to determine the performance of the model and to predict a systemic activity of the biological system it represents under changing conditions. To accomplish this the process 100 moves on to step 150.

In step 150 a simulation is formulated by specifying initial conditions and parameters to the model. A simulation is performed to determine the maximum production of metabolite E by the network under the condition that both metabolites A and C are available to be taken up by the network at a rate of 10 units/minute. Accordingly, the constraints placed on reactions A_in and C_in are:

0≦A_{—}in≦10

0<C_{—}in≦10

If there is no regulation incorporated into the model, for example, by not performing step 130 and 140, then the biochemical reaction network will utilize both A and C at the rate of 10 units/minute and maximally produce metabolite E at a rate of 10 units/minute. This is illustrated in FIG. 2C. The solution can be calculated using algorithms known in the art for linear programming.

Since there are regulatory constraints on the network, the effects of these constraints can be taken into consideration in the context of the condition being examined to determine if there are additional constraints associated with regulation that will impact the reaction network's performance. Such constraints constitute condition-dependent constraints. The process 100 thus moves to step 160, wherein the reaction constraints are adjusted based on any regulatory features relevant to the condition. In the example network in FIG. 2, there is a Boolean rule stating that if metabolite A is being taken up by the reaction network then variable Reg-R2 is 0 which means that reaction R2 is inhibited. In the condition considered in this example, A is available for uptake and will therefore inhibit reaction R2. The value for all of the regulatory Boolean reaction variables will be as follows for the specific condition considered.

Reg-R1=1

Reg-R2=0

Reg-R3=1

Reg-R4=1

Reg-A_in=1

Reg-C_in=1

Reg-E_out=1

The reaction constraints placed on each of the reactions in step 120 can then be refined using the following general equation:

$$( \begin{matrix}lower \\ bound \\ value\end{matrix} ) * ( \begin{matrix}Boolean \\ regulatory \\ variable\end{matrix} ) \leq Reaction variable \leq ( \begin{matrix}upper \\ bound \\ value\end{matrix} ) * ( \begin{matrix}Boolean \\ regulatory \\ variable\end{matrix} )$$

Examining reaction R2 in particular this equation is written as follows:

(0)*Reg-R2≦R2≦(∞)*Reg-R2

Since Reg-R2 equals zero this will change the original constraints on reaction R2 in the biochemical reaction network to be as follows:

With the effects of the regulatory network taken into consideration and the condition-dependent constraints set to relevant values, the behavior of the biochemical reaction network can be simulated for the conditions considered. This moves the process 100 to step 180. For the example model with reaction R2 now inhibited as indicated in the constraint above, metabolite C will not be taken up by the network represented therein. The maximal production of E can be calculated again through the use of linear programming leading to a value of 5 units/minute. The complete solution and flux distribution is illustrated in FIG. 2D. This is contrasted to the solution of the model without the regulatory constraints shown in FIG. 2C. The integration of regulatory constraints has reshaped the solution space for the problem and reduced the production capabilities of the example network.

The description set forth above demonstrates the general process by which regulatory constraints can be incorporated into a model of a biochemical reaction network and used to simulate the performance of a system under various conditions and concludes process 100. It is understood that other data structures that relate reactants to reactions of a reaction network such as matrices or others set forth above can be used in the process. It is also understood that other representations for regulatory reactions can be used as a function to alter the value of a variable constraint. Such representations can include, for example, fuzzy logic, heuristic rule-based descriptions, differential equations or kinetic equations detailing system dynamics.

Incorporating Molecular Mechanisms of Regulation

As exemplified above, the regulatory structure can include a general control stating that a reaction is inhibited by a particular environmental condition. Thus, it is possible to incorporate molecular mechanisms and additional detail into the regulatory structure that is responsible for determining the active nature of a particular chemical reaction within an organism. Additionally, regulation can be simulated by a model of the invention and used to predict a systemic property without knowledge of the precise molecular mechanisms involved in the reaction network being modeled. Thus, the model can be used to predict, in silico, overall regulatory events or causal relationships that are not apparent from in vivo observation of any one reaction in a network or whose in vivo effects on a particular reaction are not known. Such overall regulatory effects can include those that result from overall environmental conditions such as changes in pH, temperature, redox potential, or the passage of time.

Consider the case where a biochemical reaction network is a whole cell metabolic network, wherein the majority of the reactions are catalyzed by enzymes and proteins whose genes are encoded in the organism's genome. There is a wide range of potential mechanisms for controlling and determining the activity state of any reaction in the network. The controlling regulation can occur at various levels including, for example, transcriptional control; RNA processing control; RNA transport control (eukaryote only); translational control; mRNA degradation control or protein activity control such as activation, inhibition, phosphorylation or cofactor requirements. Collectively these regulatory reactions will determine which genes and corresponding proteins are expressed in the cell. Thus, if the required genes are present in the cell along with the required regulatory or controlling environment the associated chemical reaction can be capable of proceeding.

FIG. 3 provides a schematic drawing illustrating an example regulatory network for a reaction that includes many different types of regulatory events involved in a gene-associated reaction. These events can include, for example, inducible regulation of transcription of a protein or its subunits in the same or different operons, assembly of protein or enzyme subunits (including those encoded by both constitutively and inducibly expressed genes), or cofactor requirements for functional enzymes. Functions, such as the logic statements described above, can be included in the model to represent these regulatory events. As shown in FIG. 3, the state of the logical process (rxn_{Logic}) restrains a stoichiometric reaction by determining the condition specific constraint set to be applied to the reaction. The regulatory network shown in FIG. 3 includes regulation at the transcriptional level via transcription factors (TF) and shows constitutive expression of genes. In addition FIG. 3 shows how the process of transcription, translation, protein assembly and cofactor requirements can be incorporated into logic statements. The logical processes and functions include (a₁, a₂) for activation events, (c₁, c₂, c₃) for transcription events, (l₁, l₂, l₃) for translation events, (pi) for protein assembly and (rxn_{Logic}) for a reaction process. The memorization variables are (TF*, Mgene1, Mgene2, Mgene3, Pgene1, Pgene2, Pgene3, and Protein) corresponding to the transcription factor, mRNA transcripts, translated protein subunits, and the functional protein. The use of logic statements is described, for example, in Thomas, J. Theor. Biol.73:631-656 (1978).

Transient Implementation

The invention provides a method for determining a systemic property of a biochemical reaction network at a first and second time. The method includes the steps of (a) providing a data structure relating a plurality of reactants to a plurality of reactions of a biochemical reaction network, wherein each of the reactions includes a reactant identified as a substrate of the reaction, a reactant identified as a product of the reaction and a stoichiometric coefficient relating the substrate and the product, and wherein at least one of the reactions is a regulated reaction; (b) providing a constraint set for the plurality of reactions, wherein the constraint set includes a variable constraint for the regulated reaction; (c) providing a condition-dependent value to the variable constraint; (d) providing an objective function (e) determining at least one flux distribution at a first time that minimizes or maximizes the objective function when the constraint set is applied to the data structure, thereby determining a systemic property of the biochemical reaction network at the first time; and (f) repeating step (e), thereby determining a systemic property of the biochemical reaction network at a second time. The method can include a step of modifying the value provided to the variable constraint, for example, prior to repeating step (e).

As described above, the regulatory component of the model can be specified by the development of Boolean logic equations or a functionally equivalent method to describe transcriptional regulation as well as any other regulatory event related to metabolism. Using tanscriptional regulation as an example, transcription can be represented by the value 1 and absence of transcription can be represented by the value 0 in the constraint for a reaction that is dependent upon the transcription event. Similarly, the presence of an enzyme or regulatory protein, or the presence of certain conditions inside or outside of the cell, may be expressed as 1 if the enzyme, protein, or condition is present and 0 if it is not. The Boolean logic representation can include well-known modifiers such as AND, OR, and NOT, which can be used to develop equations governing the outcome of regulatory events.

The expression status of genes and activity of related reactions is a dynamic property within a cell. Genes are continuously being up-regulated or down-regulated as conditions are changing in the cellular environment. This situation makes regulation a transient process within the cell. To handle this situation in the regulatory structure, time delays can be introduced for each process in the logical description. Time delays can be represented by Boolean logical modeling as described in Thomas, J. Theoretical Biol. 42:563-585 (1973).

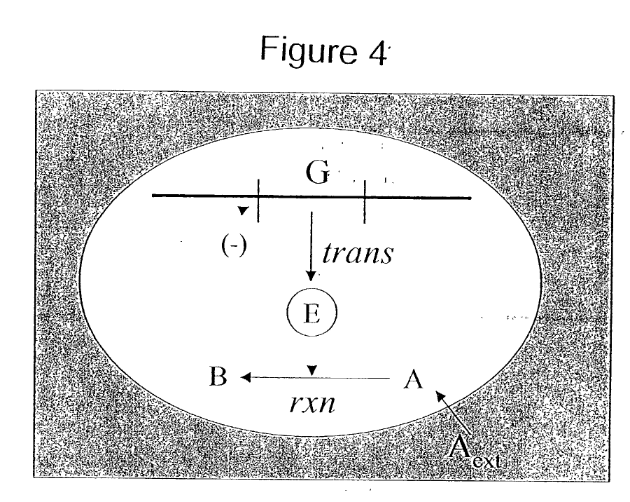

An exemplary system that can be modeled with time delays is depicted in FIG. 4. The system contains a gene G which is transcribed by a process trans, resulting in an enzyme E. This enzyme then catalyses the reaction rxn which is the conversion of substrate A to product B. The product B interacts with a binding site near G such that the transcription process trans is inhibited. In other words, the transcription event trans will occur if the gene G is present in the genome and the product B is not present to bind to the DNA. A logic equation which describes this circumstance is:

trans=IF (G) AND NOT (B)

After a certain time for protein synthesis has lapsed, progression of the transcription/translation process trans will result in significant amounts of enzyme E. Similarly, after a certain protein decay time, the absence of process trans will result in decay and eventual depletion of E.

The requirement for the reaction rxn to proceed is the presence of A and of E, for which a logical equation can be written:

rxn=IF (A) AND (E)

The presence of enzymes or regulatory proteins in a cell at a given point in time depends both on the previous transcription history of the cell and on the rates of protein synthesis and decay. If sufficient time for protein synthesis has elapsed since a transcription event for a particular transcription unit occurred, enzyme E is considered to be present in the cell. Enzyme E remains present until the time for E to decay has elapsed without the cell experiencing another transcription event for that specific transcription unit. Thus, dynamic parameters, such as the time delays of protein synthesis and degradation or causal relationships that represent regulation of gene transcription, can be included in a model of the invention. Under steady-state conditions, the average protein synthesis and degradation times are equal.

Once the presence of regulated enzymes in the metabolic network has been determined for a given time interval (t₁→t₂), if an enzyme has been determined “not present” for the time interval, then the flux through that enzyme is set to zero. This restriction may be thought of as adding a temporary constraint on the metabolic network

vₖ(t)=0, when t₁≦t≦t₂

where vₖ is the flux through a reaction at the given time point t. If an enzyme is “present” during a given time interval, the corresponding flux is left unconstrained by regulation.

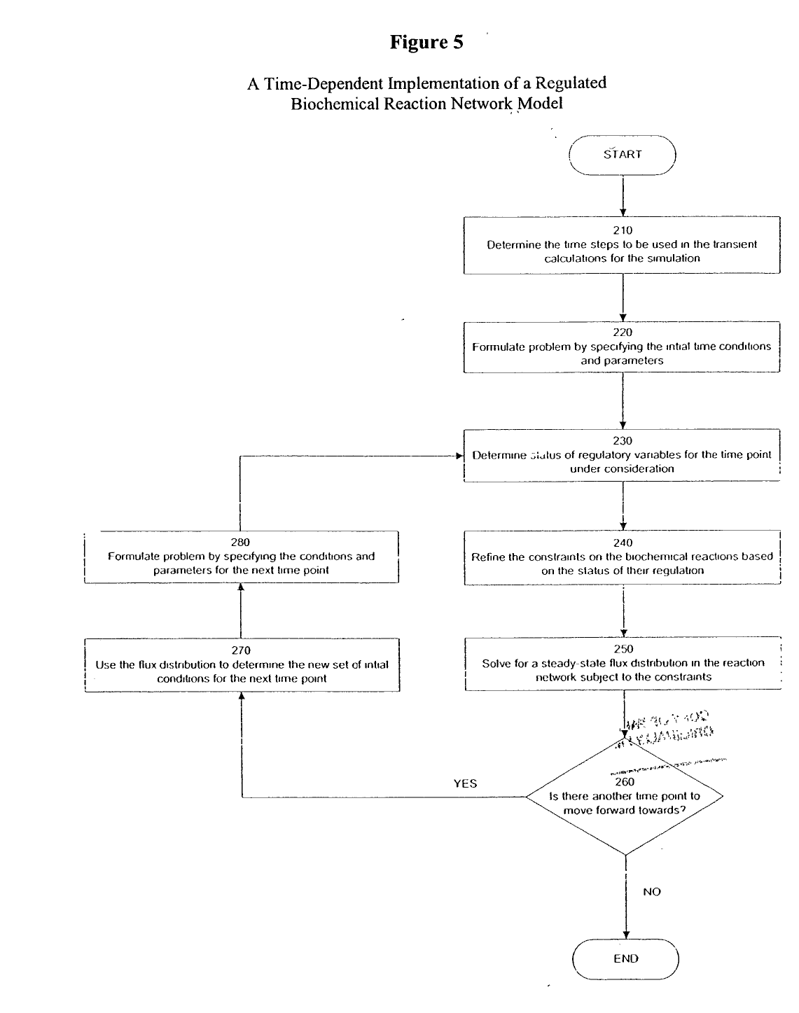

A process for the transient implementation of a biochemical reaction network model with regulation is illustrated in FIG. 5. This process 200 begins with step 210 wherein the simulation time period to be examined is first divided into a number of time steps. An example is a one hour simulation that may be divided into 10 time steps of 6 minutes each. Beginning at time zero the initial conditions for the input parameters to the regulatory structure are established in step 220 (analogous to step 150 in process 100). The process 200 then moves to step 230 (analogous to step 160 in process 100) to determine the status of the regulatory variables associated with the reactions in the biochemical reaction network model based on the input parameters established in step 220. The constraints placed on the reactions in the biochemical reaction network are then refined based on the status of the regulatory variables associated with each of the reactions in the network. This step 240 is analogous to step 170 of process 100. The process 200 then moves on to step 250 wherein a flux distribution is calculated for the reaction network analogous to step 180 of process 100. The process 200 then goes through a decision at step 260 to advance forward to the next time point if one exists. If there are no further time points then the process 200 will terminate. If there is a future time step to consider the process moves forward to step 270. In this step the initial conditions for the inputs to the regulatory structure and the initial reaction constraints are set based on the calculated solution from the previous time step as found in step 250. The problem is then fully formulated for the time point in step 280 (analogous to step 150 in process 100) wherein additional changes to the conditions can be inserted based on conditions being simulated. The process then loops through step 230, 240 and 250 to reach the decision as step 260 to continue on again to the next time point. The process 200 then will provide the complete transient response of the model to the conditions specified.

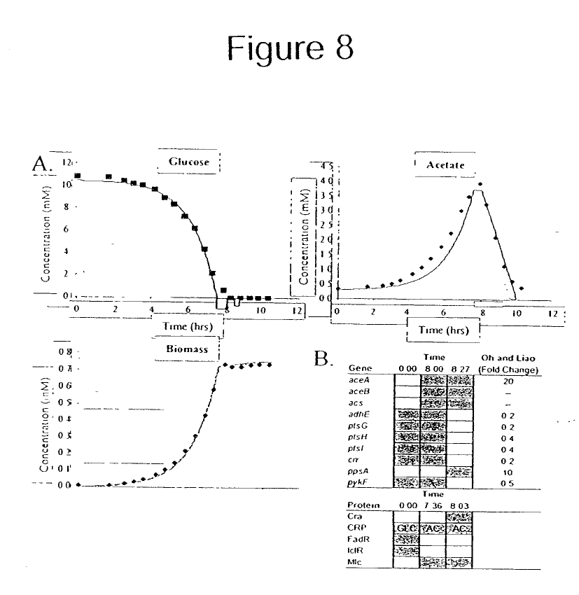

Using time delays or any other time-dependent description of the regulatory structure allows for the ability to predict the transient response of a reaction network to changing environmental conditions. This embodiment of the invention also provides a computational, as opposed to an experimental, method for the investigation of systemic responses to shifts in environmental conditions such as substrate availability or to internal changes such as gene deletion or addition. When considering a whole cell model of metabolism and regulation, this analysis can predict the transient shifts in gene expression, thus providing a computational as opposed to an experimental strategy to examine gene expression. The invention therefore provides a high-throughput computational method to analyze, interpret and predict the results of gene chip or microarray expression experiments. Use of a model of the invention to predict gene expression levels is demonstrated in Example IV and shown in Panel C of FIGS. 8, 9 and 10.

Genome Scale Implementation

Although exemplified above with regard to small reaction networks, a regulated biochemical reaction network model can be constructed and implemented for a plurality of reactions that include a plurality of regulated reactions. As used herein, the term “plurality,” when used in reference to reactions, reactants or events, is intended to mean at least 2 reactions, reactants or events. The term can include any number of reactions, reactants or events in the range from 2 to the number that naturally occur for a particular organism. Thus, the term can include, for example, at least 10, 50, 100, 150, 250, 400, 500, 750, 1000 or more reactions, reactants or events. The term can also include a portion of the total number of naturally occurring reactions for a particular organism such as at least 20%, 30%, 50%, 60%, 75%, 90%, 95% or 98% of the total number of naturally occurring reactions for a particular organism. A regulatory model that includes metabolic reactions for a whole organism or substantially all of the metabolic reactions of an organism is a genome-scale regulatory metabolic model.

In one embodiment, the invention provides a genome-scale regulatory metabolic model constructed from genome annotation data and, optionally, biochemical data. The functions of the metabolic and regulatory genes in a target organism with a sequenced genome can be determined by homology searches against databases of genes from similar organisms. Once a potential function is assigned to each metabolic and regulatory gene of the target organism, the resulting data can be analyzed. Annotation and information that can be used in this embodiment of the invention includes the genome sequence, the annotation data, or regulatory data such as the location of transcriptional units or regulatory protein binding sites, as well as the biomass requirements of an organism. Such information can be used to construct essentially genomically complete data structures representing metabolic and regulatory genotypes. These data structures can be analyzed using mathematical methods such as those described above.

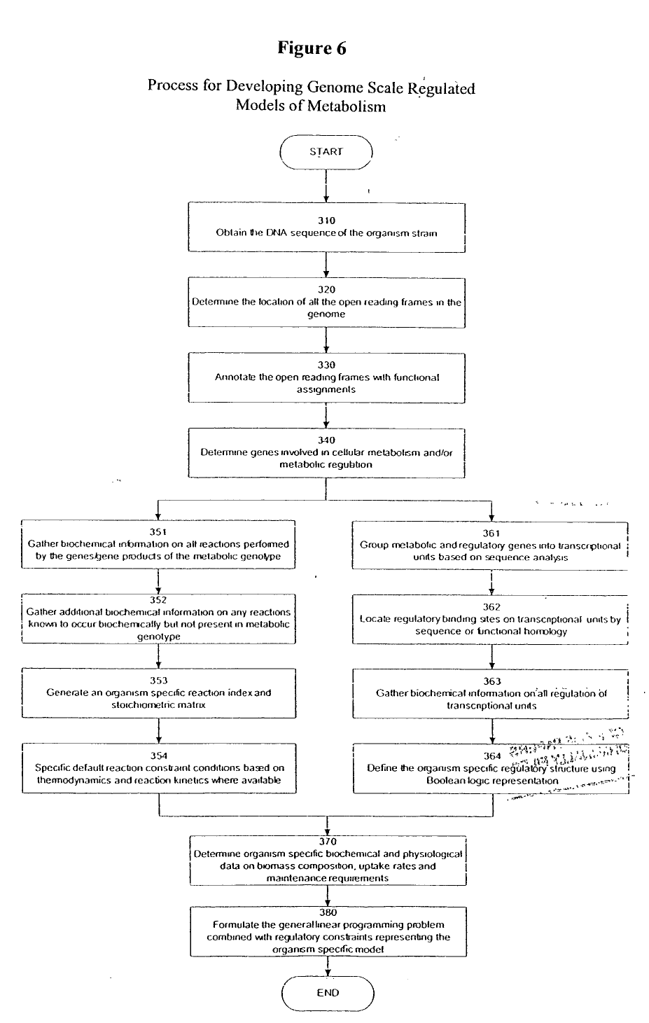

FIG. 6 shows a flow diagram illustrating a procedure for creating a genome-scale metabolic regulatory model from genomic sequence and biochemical data from an organism. This process 300 begins with step 310 by obtaining the sequenced genome of an organism. The DNA sequences of the genomes of many organisms can be found readily on public commercial databases such as The Institute for Genome Research database (TIGR), the Kyoto Encyclopedia of Genes and Genomes (KEGG) (Ogata et al., Nucleic Acids Res. 27:29-34 (1999), and many more which are now available from the private sector.

Once nucleotide sequences of the genomic DNA in the target organism have been obtained, the coding regions or open reading frames (ORFs) that encode genes from within the genome can be determined. This moves process 300 to step 320 wherein the ORFs are identified. For example, to identify the proper location, strand, and reading frame of an open reading frame one can perform a gene search by signal such as sequences for promoters or ribosomal binding sites, or by content such as positional base frequencies or codon preference. Computer programs for determining ORFs are available, for example, from the University of Wisconsin Genetics Computer Group and the National Center for Biotechnology Information.

The next step in functional annotation of a genome sequence is to annotate the coding regions or open reading frames (ORFs) on the sequence with functional assignments. This moves process 300 to step 330 to complete what is known in the art as genome annotation. Each ORF is initially searched against databases with the goal of assigning it a putative function. Established algorithms such as the BLAST or FASTA families of programs can be used to determine the similarity between a given sequence and gene/protein sequences deposited in sequence databases (Altschul et al., Nucleic Acids Res. 25:3389-3402 (1997) and Pearson et al., Genomics 46:24-36 (1997)). A large fraction of the genes for a newly-sequenced organism can usually be readily identified by homology to genes found in other organisms.

As the number of sequenced organisms rises, new techniques have been developed to determine the functions of gene products, such as gene clustering by function or by location. Several genes with related metabolic functions may be thought of as specifying a certain “pathway” which performs a certain function in a cell. Once the genes have been assigned a function by ORF homology, the genes can be categorized by pathway and comparison to other organisms can be made via available computer algorithms to locate genes which fill in pathways, etc. The comparison of relative gene location on the chromosomes in different organisms may be used to predict operon clustering. Predicted operons can be used as asserted pathways and other methods for gene functional assignments (Overbeek et al., Nucleic Acids Res. 28:123-125 (2000) and Eisenberg et al., Nature 405:823-826 (2000)).

In many cases, the functional annotation of complete and even partial or “gapped” genomes has been performed previously (Selkov et al., Proc. Natl. Acad. Sci. USA 97:3509-3514 (2000)) and can be found at websites such as the What Is There database (WIT) (Overbeek et al., Nucleic Acids Res. 28:123-125 (2000)) or KEGG.

The process 300 then moves to step 340 in which all of the genes involved in cellular metabolism and/or metabolic regulation are determined. All of the genes involved in metabolic reactions and functions in a cell comprise only a subset of the genotype. A subset of genes including genes involved in metabolic reactions and functions in a cell is referred to as the metabolic genotype of a particular organism. Thus, the metabolic genotype of an organism includes most or all of the genes involved in the organism's metabolism. The gene products produced from the set of metabolic genes in the metabolic genotype carry out all or most of the enzymatic reactions and transport reactions known to occur within the target organism as determined from the genomic sequence.

The collection of genes involved in transcriptional regulation of gene product synthesis in a cell comprises another subset of the genotype. This subset can be further reduced to incorporate those genes which regulate transcription of either a gene found in the metabolic genotype or a transcriptional regulatory gene. To begin the selection of this subset of genes, one can simply search through the list of functional gene assignments to find genes involved in cellular metabolism. This would include genes involved directly in or in the regulation of metabolic pathways such as central metabolism, amino acid metabolism, nucleotide metabolism, fatty acid and lipid metabolism, carbohydrate assimilation, vitamin and cofactor biosynthesis, energy and redox generation, or others that are described above.

The paths in the process 300 are depicted as occurring in parallel in steps 351-354 and 361-364 and respectively cover the construction of the metabolic model and regulatory model. Once these paths have been completed, the metabolic component and the regulatory component of the model are specified. These paths are described below in further detail.

Many of the organisms whose genomes have been completely sequenced to date have also been the subject of extensive biochemical research. The metabolic biochemical literature can be investigated to assign pertinent biochemical reactions to the enzymes found in the genome; to validate and scrutinize information already found in the genome; or to determine the presence of reactions or pathways not indicated by current genomic data.

In step 351, biochemical information is gathered for the reactions performed by each metabolic gene product for each of the genes in the metabolic genotype. For each gene in the metabolic genotype, the substrates and products, as well as the stoichiometry of any reactions performed by the gene product of each gene can be determined by reference to the biochemical literature or through experimental techniques. This includes information regarding the thermodynamic irreversible or reversible nature of the reactions. The stoichiometry of each reaction provides the molecular ratios in which reactants are converted into products.

Potentially, there may still remain a few reactions in cellular metabolism which are known to occur from in vitro assays and experimental data. These would include well characterized reactions for which a gene or protein has yet to be identified, or was unidentified from the genome sequencing and functional assignment. This would also include the transport of metabolites into or out of the cell by uncharacterized genes related to transport. Thus one reason for the missing gene information may be due to a lack of characterization of the actual gene that performs a known biochemical conversion. Therefore upon careful review of existing biochemical literature and available experimental data, additional metabolic reactions can be added to the list of metabolic reactions determined from the metabolic genotype. Step 352 leads to the addition of these so called non-gene associated reactions to the growing list of reactions in the model. This would include information regarding the substrates, products, reversibility irreversibility, and stoichiometry of the reactions.

The process 300 then moves to step 353 wherein the reactions postulated to occur in the organism strain based on the collective information gathered from genomic, biochemical, and physiological data is listed. This organism strain specific set of reactions is referred to as the organism specific reaction index. This reaction index contains a list of chemical reactions that are able to occur in the network. This information on reactions and their stoichiometry can be represented in a data structure of the invention such as a matrix typically referred to as a stoichiometric matrix. Each column in the matrix corresponds to a given reaction or flux, and each row corresponds to the different metabolites involved in the given reaction/flux. Reversible reactions can ether be represented as one reaction that operates in both the forward and reverse direction or can be decomposed into one forward reaction and one backward reaction in which case all fluxes can only take on positive values. Thus, a given position in the matrix describes the stoichiometric participation of a metabolite (listed in the given row) in a particular flux of interest (listed in the given column). Together all of the columns of the genome specific stoichiometric matrix represent all of the chemical conversions and cellular transport processes that are determined to be present in the organism. This includes all internal fluxes and so called exchange fluxes operating within the metabolic network. The resulting organism strain specific stoichiometric matrix is a fundamental metabolic representation of a genomically and biochemically defined organism.

Constraints can be placed on the reactions based on the thermodynamics of the reactions and additional biochemical information that is required. These constraints can be referred to as “default constraints” placed on reactions in a general problem formulation and are specified in step 354. All of the reactions in the network can be constrained with an upper and a lower bound. These bounds can be finite numerical values, zero or values of negative or positive infinity. For a reversible reaction the lower bound would be set to negative infinity and the upper bound set to positive infinity. These sets of bounds would effectively make the reaction unconstrained in terms of its flux level. Alternatively a reaction may be irreversible in which case the lower bound would be zero and the upper bound would be positive infinity, thereby forcing the reaction to take on a positive flux value. If information regarding the maximum flux capacity of a reaction is available, the upper bounds can be specified to equal this maximum capacity, which will serve to further constrain the allowable flux levels of the reactions.

With the completion of step 354 the construction of the metabolic portion of the model is completed. In parallel the regulatory portion of the model is also constructed as detailed in steps 361 to 364 described below.

Two potential approaches that can be used in constructing the regulatory structure are the “bottom-up” and the “top-down” approaches. In the “bottom-up” approach, the biochemical literature is searched to determine transcription units, which can be a single gene or a group of genes which are transcribed as a unit. This can be determined from the biochemical literature, or using bioinformatics techniques such as sequence analysis to find promoter regions by homology or the like (Ermolaeva et al., Nucleic Acids Res. 29:1216-1221 (2001)). Databases such as RegulonDB make this information available to the public for commonly studied organisms (Salgado et al., Nucleic Acids Re. 29:72-74 (2001)).

The transcriptional units of the organism can then be located. This may be done by sequence analysis, for example, by locating putative promoter binding sequences on a bacterial genome and grouping genes by functional assignment and location or by studying the biochemical literature. In step 361 of process 300 the metabolic and regulatory genes to be considered in the regulatory component of the model are identified as transcriptional units.

The transcriptional regulation of identified transcription units can be further investigated using the biochemical literature and/or databases. Each transcription unit may be regulated by one or more regulatory mechanisms, or may be constitutively expressed. Proteins generally bind to a site on the DNA where they may either repress or activate transcription of the transcription unit. These binding sites may be identified for a particular genome sequence by homology with known binding sites. Furthermore, such binding sites and regulatory proteins may be investigated experimentally to determine such characteristics as the nature of regulation such as repression or activation, for each regulatory protein; the binding affinity of each regulatory protein to the appropriate binding site or the cooperation/interaction of co-regulatory proteins to regulate expression of a particular transcription unit.

The identification of these regulatory binding sites on transcriptional units by sequence analysis or functional homology represents step 362 of process 300. Thus, the initial process of determining how the reactions in the metabolic network are regulated can occur by determining the association of transcriptional units with predicted regulatory events. To complete the determination, step 363 can be performed wherein the actual biological method of regulation of the transcriptional units is elucidated in so far as it is known. In addition, any regulation associated with events that are independent of transcription, such as enzymatic inhibition or enzyme cofactor requirements, can be gathered at this step to add further information to the regulatory structure.

An alternative approach to elucidating the regulatory structure described in steps 361 to 363 involves expression profiling or similar technologies implemented to determine which genes are actually being used under a particular physiological condition, and methods of systems identification, to phenomenologically and systematically find relationships between the expressed genes. The use of expression profiling and systems identification can thus be used to find groups of genes, associated reactions, or even extreme pathways that are operational under the physiological conditions of interest through an approach that essentially involves a “top-down” approach since the behavior of the entire system is measured at once. The “top-down” or “bottom-up” approaches may be used separately or in combination to define the regulatory structure of an organism on a genome scale.

With the biological regulatory mechanisms and phenomena identified for inclusion into the model, the process 300 then moves to step 364 wherein the regulatory structure is represented mathematically in a data structure for integration with the metabolic component of the model. The regulatory component of the model can be specified by the development of Boolean logic (or equivalent) equations to describe transcriptional regulation as well as any other regulatory event related to metabolism. This involves restricting expression of a transcription unit to the value 1 if the transcription unit is transcribed and 0 if it is not. Similarly, the presence of an enzyme or regulatory protein, or the presence of certain conditions inside or outside of the cell, may be expressed as 1 if the enzyme, protein, or condition is present and 0 if it is not. The synthesis time of a protein from a particular transcription unit may be determined experimentally, from the biochemical literature, or estimated by similarity to other proteins. Additional time dependencies between regulatory parameters can be specified and delays introduced in the regulatory structure.

At this point in the process 300 the metabolic and regulatory networks have been developed and described mathematically to allow for their integrated analysis. Common approaches used to study the metabolic network without regulatory constraints can still be used to assess the affect of the constraints that regulation now places on metabolism. An example of this is to combine the regulatory structure with pathway analysis to examine the effects of regulation on the solution space. Pathway analysis uses principles of convex analysis to study the characteristics of the solution space. The extreme pathways calculated by pathway analysis are edges of the solution space where the optimal solution must lie (Schilling et al., J. Theor. Biol. 203:229-258 (2000)). The extreme pathways that describe the capabilities of the metabolic network are calculated by determining a set of vectors that span the solution space. Each vector represents an extreme pathway (Schilling et al., Biotech. Bioeng. 71:286-306 (2000)). The algorithm used to generate these vectors has recently been described in detail (Schilling et al., J. Theor. Bio. 203:229-248 (2000)). For a given environment, the corresponding regulatory constraints are determined (e.g., repression of gene transcription) and extreme pathways that are inconsistent with the imposed regulatory constraints are eliminated. This procedure reduces the solution-space and customizes it for the given circumstance serving as a method of model reduction.

In process 300, the integrated regulatory and metabolic network is examined through the use of flux balance analysis to study the optimal metabolic properties of the organism. This moves the process 300 to step 370 where a collection of organism specific biochemical and physiological data is gathered. These data can include the biomass compositions, uptake rates, and maintenance requirements of the organism under various environmental conditions. Experiments can be performed to determine the uptake rates and maintenance requirements for the organism or, alternatively, these values can be obtained from the literature. The uptake rate for metabolites transported into the cell can be determined experimentally by measuring the depletion of the substrate from the growth media. A measurement of the biomass at each time point can also be made, in order to determine the uptake rate per unit biomass. The maintenance requirements can be determined from a chemostat experiment. For example, the glucose uptake rate can be plotted versus the growth rate, and the y-intercept interpreted as the non-growth associated maintenance requirements. The growth associated maintenance requirements can be determined by fitting the model results to the experimentally determined points in a growth rate versus glucose uptake rate plot.

Additionally, the metabolic demands placed on the organism can be determined. The metabolic demands can be readily determined from the dry weight composition of the cell when cell growth is the objective function under consideration. In the case of well-studied organisms, such as E. coli and Bacillus subtilis, the dry weight composition is available in the published literature. However, in some cases it will be necessary to experimentally determine the dry weight composition of the cell for the organism in question. This can be accomplished for various components of the cell, including RNA, DNA, protein, and lipid, with a more detailed analysis providing the specific fractions of nucleotides, amino acids, etc.

With sufficient biochemical and physiological data provided, appropriate constraints can be specified for the relevant reactions and growth related demand fluxes are put in place. This leads to the complete formulation of a general problem to be solved regarding the organism using the integrated regulatory metabolic model. This moves process 300 to step 380 wherein the general linear programming problem forming the basis of a flux balance analysis is formulated based on the combined metabolic and regulatory constraints. This is discussed below -in detail.

The time constants characterizing metabolic transients and/or metabolic reactions are typically very rapid, on the order of milli-seconds to seconds, compared to the time constants of cell growth on the order of hours to days (McAdams and Arkin, Ann. Rev. Biophy. Biomol. Struc. 27:199-224 (1998)). Thus, the transient mass balances can be simplified to only consider the steady state behavior. Eliminating the time derivatives obtained from dynamic mass balances around every metabolite in the metabolic system, yields a system of linear equations represented in matrix notation,

S·v=0

where S refers to the stoichiometric matrix of the system, and v is the flux vector. This equation simply states that over long times, the formation fluxes of a metabolite must be balanced by the degradation fluxes. Otherwise, significant amounts of the metabolite will accumulate inside the metabolic network. Applying this equation to a biological system, S represents the system specific stoichiometric matrix generated from the reaction index.

To determine the metabolic capabilities of a defined metabolic genotype the above equation is solved for the metabolic fluxes and the internal metabolic reactions, v, while imposing constraints on the activity of these fluxes. Typically the number of metabolic fluxes (n) is greater than the number of mass balances or metabolites (m) (i.e., n>m) resulting in a plurality of feasible flux distributions that satisfy this equation and any constraints placed on the fluxes of the system. This range of solutions is indicative of the flexibility in the flux distributions that can be achieved with a given set of metabolic reactions. The solutions to this equation lie in a restricted region. This subspace defines the capabilities of the metabolic genotype of a given organism, since the allowable solutions that satisfy this equation and any constraints placed on the fluxes of the system define all the metabolic flux distributions that can be achieved with a particular set of metabolic genes.

The particular utilization of the metabolic genotype can be defined as the metabolic phenotype that is expressed under those particular conditions. Objectives for metabolic function can be chosen to explore the ‘best’ use of the metabolic network within a given metabolic genotype. The solution to the above equation can be formulated as a linear programming problem, in which the flux distribution that minimizes a particular objective is found. Mathematically, this optimization can be stated as;

Minimize Z

subject to Z=cᵢ×vᵢ=(c·v)

Where Z is the objective which is represented as a linear combination of metabolic fluxes vᵢ. The optimization can also be stated as the equivalent maximization problem; i.e. by changing the sign on Z.

This general representation of Z enables the formulation of a number of diverse objectives. These objectives can be design objectives for a strain, exploitation of the metabolic capabilities of a genotype, or physiologically meaningful objective functions, such as maximum cellular growth. For this application, growth is to be defined in terms of biosynthetic requirements based on literature values of biomass composition or experimentally determined values. Thus, biomass generation can be described as an additional reaction flux draining intermediate metabolites in the appropriate ratios and represented as an objective function Z. In addition to draining intermediate metabolites this reaction flux can be formed to utilize energy molecules such as ATP, NADH and NADPH so as to incorporate any maintenance requirement that must be met. This new reaction flux then becomes another constraint/balance equation that the system must satisfy as the objective function. It is analogous to adding an additional column to the stoichiometric matrix S to represent such a flux to describe the production demands placed on the metabolic system. Setting this new flux as the objective function and asking the system to maximize the value of this flux for a given set of constraints on all the other fluxes is then a method to simulate the growth of the organism.

Using linear programming, additional constraints can be placed on the value of any of the fluxes in the metabolic network, as described above, in the form of:

βⱼ≦vⱼ≦αⱼ

These constraints could be representative of a maximum allowable flux through a given reaction, possibly resulting from a limited amount of an enzyme present in which case the value for αⱼ would take on a finite value. These constraints could also be used to include the knowledge of the minimum flux through a certain metabolic reaction in which case the value for βⱼ would take on a finite value. Additionally, if one chooses to leave certain reversible reactions or transport fluxes to operate in a forward and reverse manner the flux may remain unconstrained by setting βⱼ to negative infinity and αⱼ to positive infinity. If reactions proceed only in the forward reaction βⱼ is set to zero while αⱼ is set to positive infinity.

This step of assigning these basic constraints to the values of reactions is what occurs in step 354 of process 300. These constraints can be further refined based on specific environmental or genetic conditions that are to be examined for the problem of interest being formulated in step 380. As an example, to simulate the event of a genetic deletion the flux through all of the corresponding metabolic reactions related to the gene in question are reduced to zero by setting βⱼ and αⱼ to zero in the above equation.

Based on the in vivo environment of the organism, one can determine the metabolic resources available for biosynthesis of essential molecules for biomass. Allowing the corresponding transport fluxes to be active provides the in silico organism with inputs and outputs for substrates and by-products produced by the metabolic network. Therefore, as an example, if one wished to simulate the absence of a particular growth substrate one simply constrains the corresponding transport fluxes allowing the metabolite to enter the cell to be zero by allowing βⱼ and αⱼ to be zero. On the other hand if a substrate is only allowed to enter or exit the cell via transport mechanisms, the corresponding fluxes can be properly constrained to reflect this scenario.

Together the linear programming representation of the genome-specific stoichiometric matrix along with any general constraints placed on the fluxes in the system, and any of the possible objective functions completes the formulation of the in silico metabolic model. The in silico model can then be used to predict metabolic capabilities by simulating any number of conditions and generating flux distributions through the use of linear programming. With the incorporation of the regulatory constraints as discussed in process 100 the model can be used to explore metabolic performance issues that have been intractable based on the current art of constraints-based modeling without any representation of regulation or to reduce the solution space thereby increasing the predictive power of constraints-based models.

Once the models have been constructed, they may be used to generate dynamic profiles of a phenotype using a procedure such as the one described in process 200. This approach can be used, for example, for calculating dynamic gene expression, metabolic fluxes, and extracellular substrate/by-product concentrations from the combined metabolic/regulatory model.

For the prediction of the time profiles of consumed and secreted metabolites, as well as gene expression profiles, in batch experiments, the experimental time may be divided into small time steps, Δt (Varma and Palsson, Biotechnology 12:994-998 (1994) and Varma and Palsson, Applied Environ. Micro. 60:3724-3731 (1994)). Beginning at t=0 where the initial conditions of the experiment are specified, the combined regulatory/metabolic model may be used to predict concentrations and gene expression for the next step as discussed in process 200. The initial conditions of the cell are determined by the conditions of an experiment or by the previous conditions of the computer simulation. Conditions such as the extracellular substrate concentrations or biomass concentration can be found experimentally. The initial presence or absence of regulatory proteins may be found experimentally (i.e. by using microarrays or gene chip technology), or by considering the steady-state solution of the Boolean logic equations.

Transcription and metabolic regulation can be described using a Boolean representation as described above. The status of transcription is found from the given conditions at the particular time interval. Specifically, transcription may be altered by the presence or surplus of an intracellular metabolite, an extracellular metabolite, regulatory proteins, signaling molecule, or any combination of these or other factors. The logical equation governing transcription of each transcriptional unit can be used to determine whether transcription occurs or does not occur.

The presence of enzymes or regulatory proteins in the cell depends on the previous transcription history of the cell and the rates of protein synthesis and decay. If the time required for protein synthesis has elapsed since a transcription event for a particular transcription unit occurred, the protein(s) are considered to be present in the cell and to remain present in the cell until the time for the protein(s) to decay has elapsed without the cell experiencing another transcription event for that specific transcription unit.

Once the presence of all regulated enzymes in the metabolic network has been determined for a given time interval, the constraints on the reactions in the metabolic component of the model are altered to reflect the temporary effects of regulation. The time constants characterizing metabolic transients and/or metabolic reactions are often orders of magnitude more rapid than those characterizing transcriptional regulation, so during each time interval a quasi steady-state can be assumed to exist where the stoichiometric matrix is constant.

The extreme pathways which define the solution space for an organism may be recalculated once these temporary constraints have been imposed to determine a new volume and dimensionality of the space. This results in the generation of a biologically meaningful subset of the original solution space, which may contain only a small fraction of the behaviors previously available to the cell.

Once the constraints imposed by regulation have been determined and applied, the concentration of all available substrates can be scaled to determine the amount of substrate available per unit of biomass per unit of time (millimoles per gram dry weight per hour) using the following equation:

$$Substrate available = \frac{{S}_{c}}{X \cdot Δ t}$$

where S_{c} is the substrate concentration and X is the cell density. If the substrate available is greater than the maximum uptake rate, the maximum uptake rate is used. The flux balance model then determines the actual substrate uptake Sᵤ as well as the growth rate μ and potential by-product secretion, as has been explained.

Once the metabolic flux distribution has been calculated using flux balance analysis, the intracellular conditions for the next time step can be determined from the flux distribution, and the extracellular substrate concentrations for the next time step can be determined from standard differential equations:

$$\frac{ⅆ X}{ⅆ t} = μ \cdot X \to X = {X}_{0} \cdot {e}^{μ \cdot Δ t}$$

$$\frac{\partial {S}_{c}}{\partial t} = - {S}_{u} \cdot X \to {S}_{c} = {S}_{c0} + \frac{{S}_{u}}{μ} {X}_{0} ( 1 - {e}^{μ \cdot Δ t} )$$

These conditions will then be used for the next time point. This provides one type of problem, namely a transient examination of the metabolic performance of an organism, that can be formulated in step 380 of process 300 covering the development and implementation of an organism specific genome scale regulated model of metabolism. The completion of step 380 concludes the process 300.

As set forth above, integrating flux-balance analysis and the relevant regulatory constraints provides a method for simulating gene expression and cellular metabolism under a wide range of conditions. The process described above can be embodied in whole or in part in a software application that can be used to create the regulatory/metabolic genotype for a fully sequenced and annotated genome. Additionally, the software application can be used to further analyze and manipulate the network so as to predict the ability of an organism to produce biomolecules necessary for growth under various conditions and thereby simulate gene expression patterns and the resultant shift in metabolic fluxes as demonstrated in the Examples below.

The recent development of experimental techniques such as microarray and gene chip technology has made it possible to determine the gene expression of an entire organism under given conditions. The ability to predict and simulate gene expression at a similar scale will advance the development and use of these new technologies. The models of the invention are able to predict gene transcriptional shifts in E. coli under a wide variety of conditions which may be directly compared to experimental gene array data as described in the Examples and shown in Panel C of FIGS. 8 through 10. The combined regulatory/metabolic model described here can qualitatively predict shifts in gene expression, producing in silico expression arrays.

An advantage of the invention is that it can be used where genome data is available for a newly discovered organism, such as a pathogen, and functional data is limited or unavailable. In this case, the ability to learn about the physiology of the particular organism and explore its metabolic capabilities without any specific biochemical data will become very important.

Although exemplified herein with respect to E. coli, the models and methods of the invention can be applied to any organisms for which biochemical or genome sequence information is available. For example, a model of Haemophilus influenzae (a respiratory pathogen) can be constructed by homology to E. coli. The metabolic network and data structure representing the network can be constructed from the genome sequence as has been described. The regulatory proteins can also be determined by homology to regulatory proteins in other organisms, and the transcriptional units and regulatory protein binding sites can be identified as has been described.

Once the above information has been determined, regulatory logic can be inferred by homology to a model from another organism such as the E. coli model exemplified above, as well as from the location of regulatory binding sites and transcriptional units. From the resultant combined regulatory/metabolic model for the organism, metabolic and gene expression shifts can be analyzed, interpreted and predicted using methods similar to those exemplified herein with respect to E. coli or model pathways.

Furthermore, it is contemplated that combined regulatory/metabolic models can be generated for multiple organisms using microarray data. In this case, the regulatory network generated from the array data can be incorporated into existing models. Furthermore, the microarray data and the available literature can be used together to reconstruct the regulatory network.

Any prokaryote, archae or eukaryote for which sequence and or biochemical information is present can be modeled according to the invention. Examples of other organisms that can be simulated by the models and methods of the invention include Bacillus subtilis, Saccharomyces cerevisiae, Haemophilus influenzae, Helicobacter pylori, Drosophila melanogaster or Homo sapiens.

The incorporation of a regulatory structure with flux balance analysis and linear optimization can also be used to simulate the activity or function of other biological networks. Those skilled in the art will be able to apply the above-described models and methods in order to simulate a variety of biological networks including, for example, networks of a cell, group of cells, organ, organism or ecosystem. Activities for individual steps or processes in the network can be converted into a data structure that relates the particular step or process to the components they act upon. In addition, the activities can be constrained using constraint sets as described above. As an example, the methods can be used to simulate a signal transduction system as a flux of free energy through the system where interactions between signaling partners are represented as reactions and are constrained with respect to the amount of energy that flows from one partner to another. Regulation can be incorporated by varying the constraints with respect to effects of cross talk between signaling systems. Similarly, physiological systems can be simulated by creating data structures that correlate physiological functions with particular organs, tissues or cells and regulatory data structures or events can be incorporated to represent the effects of stimuli or insults such as hormones, pathogens or environmental conditions that affect the physiological system. Another example, is an ecosystem for which a data structure can be constructed that relates organisms and ecological processes, wherein regulation can include a representation of changes in environmental conditions.

The following examples demonstrate the construction and implementation of combined regulatory/metabolic model, and provide experimental validation of the model predictions. The following examples are intended to illustrate but not limit the invention.

### EXAMPLE I

### Pathway Reduction in an Exemplary Metabolic Model

This example describes construction of a skeleton metabolic model having regulatory constraints. This example demonstrates that the inclusion of regulatory constraints in a flux balance analysis simulation increases the predictive ability of a skeleton metabolic model by reducing the size and dimensionality of the mathematical solution space produced by the model.

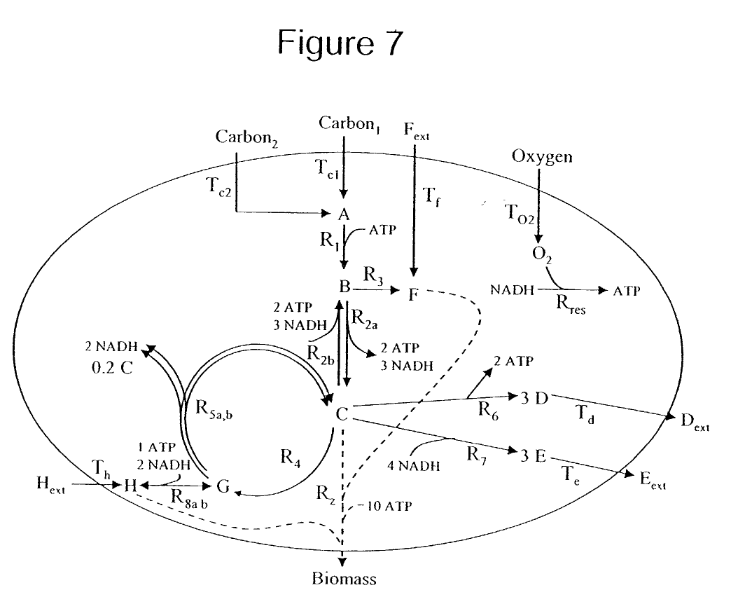

A skeleton of the biochemical reaction network of core metabolism was formulated, including 20 reactions, 7 of which are regulated as shown in the upper panel of FIG. 7. This network provided a simplified representation of core metabolic processes including glycolysis, the pentose phosphate pathway, TCA cycle, fermentation pathways, amino acid biosynthesis and cell growth, along with corresponding regulation pathways including catabolite repression, aerobic/anaerobic regulation, amino acid biosynthesis regulation and carbon storage regulation. The skeleton biochemical reaction network was represented as a skeleton combined regulatory/metabolic model where reactions were represented as linear equations of reactants and stoichiometric coefficients and regulation was represented by regulatory logic statements as shown in the lower panel of FIG. 7. As shown in FIG. 7, four regulatory proteins (Rpo2, RPc1,RPh and RPb) regulated 7 of the 20 reactions in the skeletal network and model.

The skeleton combined regulatory/metabolic model was analyzed using extreme pathway analysis. Using known algorithms, 80 extreme pathways were calculated for the given sample system by considering the metabolic reactions in the network (Schilling et al., J. Theor. Biol. 2203:229-248 (2000)). Given the five inputs to the metabolic network and representing these inputs using Boolean logic, considering each as ON if present or OFF if absent, there are a total of 2⁵⁼³² possible environments which may be recognized by the cell. These environments are listed in Table 1. For each environment, the transcription of several of the enzymes in the network may be restricted due to regulation. The constraints imposed on the system by (a) the substrates available to the cell (external environment) and (b) the enzymes present in the cell (internal environment), reduced the number of extreme pathways available to the model at a given time. Table 1 shows that the highest number of pathways available to the model was 26; the lowest was 2. This corresponded to a reduction in the number of extreme pathways in a solution space of between 67.5% to 97.5% compared to the same model where none of the reactions is subject to regulatory constraints.

These results demonstrate that the inclusion of regulatory constraints in a flux balance analysis simulation reduces the size and dimensionality of the mathematical solution space and subsequently reduces the capabilities of the metabolic network due to the imposition of additional constraints.

### EXAMPLE II

### E. coli Metabolic and Regulatory Genotype and in silico Model

This example demonstrates construction of a genome-scale combined regulatory/metabolic model for Escherichia coli K-12.

The annotated sequence of the Escherichia coli K-12 genome was obtained from Genbank, a site maintained by the NCBI (ncbi.nlm.gov). The annotated sequence included the nucleotide sequence as well as the open reading frame locations and assignments. Such annotated sequences can also be obtained from other sources such as The Institute for Genomic Research (tigr.org). From the annotated sequence, the genes involved in cellular metabolism and/or metabolic regulation were identified. A core combined regulatory/metabolic model of Escherichia coli K-12 was created by including reactions associated with genes that are annotated as being involved in cellular metabolism or metabolic regulation or both.

A detailed search of the biochemical literature was made to further develop the model. Any additional reactions known to occur from biochemical data which were not represented by the genes in the metabolic genotype were added to the Escherichia coli K-12 combined regulatory/metabolic model.

Additional, transcription units and regulatory protein binding sites were identified using the biochemical literature and online resources dealing with E. coli regulation such as that available at tula.cifn.unam.mx:8850/regulondb/regulon intro.frameset (Salgado et al., Nucleic Acids Res. 29:72-74 (2001)). The nature of the regulation of each transcription unit was determined based on the biochemical literature. The regulatory information was incorporated into a genome specific regulatory structure using a Boolean logic representation for each reaction.

The resulting E. coli K-12 core metabolic/regulatory model represented the products of 149 genes, including 16 regulatory proteins and 73 enzymes, which catalyze 113 reactions. The synthesis of 43 of the enzymes that were included in the model was found to be controlled by transcriptional regulation based on genome sequence annotation and the biochemical literature; as a result, the availability of 45 of the reactions to the model was controlled by a logic statement. Further details of the combined regulatory/metabolic network are shown in Table 2, which lists the metabolic reactions and regulatory rules for a central E. coli system.

The uptake rates and maintenance requirements for E. coli were obtained from the published literature and incorporated as exchange reactions in the model. The resulting in silico model represented the core metabolic capabilities of E. coli and the transcriptional regulation of these capabilities. In the case of E. coli K-12, the wealth of data on overall metabolic behavior and detailed biochemical information about the in vivo genotype can be utilized in order to evaluate the predictive capabilities of the in silico model as demonstrated below.

### EXAMPLE III

### Mutant Knockout Simulations

This example describes use of a stand-alone metabolic model and a combined regulatory/metabolic model for in silico prediction of growth for various E. coli mutants on different carbon sources. This example demonstrates that the in silico metabolic models can predict the growth phenotype observed in vivo for a majority of the mutants tested and that incorporation of regulation into the metabolic model increases the predictive abilities of the metabolic model.

The combined regulatory/metabolic model described in Example 2 was used to ascertain the ability of mutant strains of E. coli to grow on defined media. A similar model lacking the regulatory logic was also produced and is referred to as the stand-alone metabolic model. In each case, predictions of the combined regulatory/metabolic model or the stand-alone metabolic model were compared with experimental data from the literature. Table 3 shows results of the comparison scored as “+” for growth or “−” for no growth and presented in the order of (in vivo observations)/(stand-alone metabolic model)/(combined regulatory/metabolic model). An ‘N’ indicates that the data was not available for these conditions. Cases where the combined regulatory/metabolic model makes a correct prediction either unpredicted or incorrectly predicted by the stand-alone metabolic model are denoted by a shaded box. Rows represent a particular mutant and columns represent results for growth on a particular carbon source where “glc” is glucose, “gl” is glycerol, “suc” is succinate, “ac” is acetate, “rib” is ribose, and “(—O2)” is anaerobic conditions.

As shown in Table 3, the growth results predicted by the in silico stand-alone metabolic model correlated with empirically determined in vivo results from the literature for 83.6% of the mutants (97 of the 116 cases that were simulated). Incorrect predictions were made for 16 of the 16 cases. Predictions were not possible for 3 cases related to the rpiR mutant because rpiR is a regulatory gene and, therefore, was not included in the stand-alone metabolic model.

The combined regulatory/metabolic model made correct predictions about growth characteristics in 91.4% of the mutants (106 of the 116 cases that were simulated), yielding an improvement of 9 correct predictions over the unregulated, stand-alone metabolic model. The mutants whose growth capabilities were correctly predicted by the former model, but not the latter model were aceEF, fumA, ppc, rpiA, and rpiR. The remaining incorrect predictions are shown in Table 3 and in most cases were due to accumulation of toxic substances, an effect that was not accounted for in the combined regulatory/metabolic model.

The combined regulatory/metabolic model was used to examine in more detail the 9 mutants that were differentially predicted by the two models. According to the predictions of the combined regulatory/metabolic model, pyruvate dehydrogenase, encoded by the aceEF-lpdA operon, is a lethal mutation in E. coli for growth on minimal glucose and minimal succinate media under aerobic conditions due to the aerobic down-regulation of its fermentative counterpart, pyruvate formate-lyase. Similarly, fumarase A (fumA) is the only fumarase which is generally transcribed under aerobic conditions. Phosphoenolpyruvate carboxylase (ppc) was correctly predicted to be a lethal mutation due to the down-regulation of the glyoxylate shunt.

The ribose phosphate isomerase A (rpiA) and the ribose repressor protein RpiR illustrate how regulatory gene mutant phenotypes can be simulated using the combined regulatory/metabolic model. Two isomerases exist in E. coli for the interconversion of ribulose 5-phosphate and ribose 5-phosphate, encoded for by the rpiA and rpiB genes. While the expression of rpiA is thought to be constitutive, expression of rpiB occurs in the absence of RpiR, which is inactivated by ribose. As a result, rpiA mutants are ribose auxotrophs while rpiB mutants exhibit a null phenotype. The further mutation of rpiR in rpiA mutants disables repression of rpiB and restores the ability to grow in the absence of ribose, as correctly predicted by the combined regulatory/metabolic model.

These results demonstrate that the imposition of regulatory constraints on the solution space of an organism's metabolism result in a more accurately constrained space. This improved accuracy allowed for the correction of 9 false predictions made by the stand-alone metabolic model. Furthermore, such constraints allow accurate prediction of the phenotype for regulatory gene mutations, as demonstrated by the three rpiR mutant growth predictions made by the combined regulatory/metabolic model.

### EXAMPLE IV

### Metabolic Shifts and Associated Regulation

This example demonstrates use of the combined regulatory/metabolic model to simulate growth of E. coli quantitatively over the course of growth experiments. This example also demonstrates comparison of the resulting time courses of growth, substrate uptake, and by-product secretion to experimental data.

E. coli has been observed in vivo to secrete acetate when grown aerobically on glucose in batch cultures; when glucose is depleted from the environment, the acetate is then reutilized as a substrate. Using the combined regulatory/metabolic and stand-alone metabolic models, activity of an aerobic batch culture of E. coli on glucose minimal medium was simulated. Panel A of FIG. 8 shows three time plots showing experimental data (closed squares) and the corresponding simulations performed using the combined regulatory/metabolic model (solid lines) as well as the stand-alone metabolic model (dashed lines). In the acetate plot, the regulatory/metabolic model predictions differed from that of the stand-alone metabolic model, as shown. Panel B of FIG. 8 shows a table containing the parameters required to generate the time plots where parameters were estimated or obtained from Varma and Palsson Appl. Env. Micro. 60:3724-3731 (1994). The major difference between the combined regulatory/metabolic and metabolic stand-alone simulations is in the delayed reaction of the system to depletion of glucose in the growth medium. The stand-alone metabolic network is unable to account for the delays associated with protein synthesis.

Panel C of FIG. 8 shows In silico predictions of up- or down-regulation of selected genes, or activity of regulatory proteins, in the regulatory network represented in an array format (dark—gene transcription/protein activity, light—transcriptional repression/protein inactivity). The regulation of catabolite repressor protein (CRP) is represented by the set of Boolean statements provided in Table 2. CRP activity is represented in FIG. 8 as GLC or AC to denote when glucose or acetate is accepted by the system, respectively. The in silico array predicted the up-regulation of 4 gene products, aceA, aceB, acs, and ppsA, as well as the down-regulation of 3 gene products, adhE, ptsGHI-crr, and pykF. DNA microarray technology has been used to detect differential transcription profiles on a collection of 111 genes in E. coli as described in Oh and Liao, Biotech. Prog. 16:278-286 (2000) and the difference in gene expression for aerobic growth on acetate versus growth on glucose as reported therein is included in FIG. 8C. The eight genes included in the combined regulatory/metabolic model for which expression data was published are in qualitative agreement with the predictions of the combined regulatory/metabolic model. The ability of the combined regulatory/metabolic model to reutilize acetate depends on the up-regulation of the glyoxalate shunt genes, aceA and aceB which provides an explanation for the high magnitude of transcription difference (20-fold) reported in Oh and Liao, Biotech. Progress 16:278-286 (2000).

Furthermore, the combined regulatory/metabolic model suggested an interpretation for the regulation of two genes which were known to be regulated but by unknown causes, ppsA and adhE. The combined regulatory/metabolic model indicated that a second regulatory shift is induced by the catabolite activator protein Cra, which responds to falling intracellular concentrations of fructose 6-phosphate and fructose 1,6-bisphosphate once glucose is depleted from the medium. This second regulatory shift is responsible for the upregulation of ppsA and adhE, according to the combined regulatory/metabolic model.

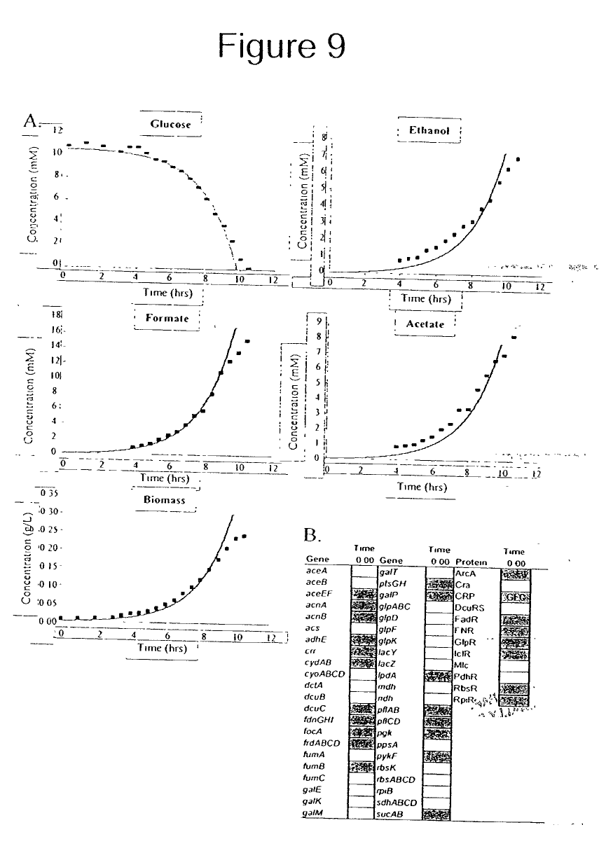

The in silico models were used to simulate anaerobic growth on glucose, the results of which are shown in FIG. 9. Under these conditions, the stand-alone metabolic model made similar predictions as the combined regulatory/metabolic model, with a notable exception: the combined regulatory/metabolic model was able to make predictions about the use of a particular isozyme. For example, both models require fumarase activity as part of the optimal flux distribution; however, of the two models only the combined regulatory/metabolic model was able to specifically determine that the fumB gene product which as being expressed under anaerobic conditions.

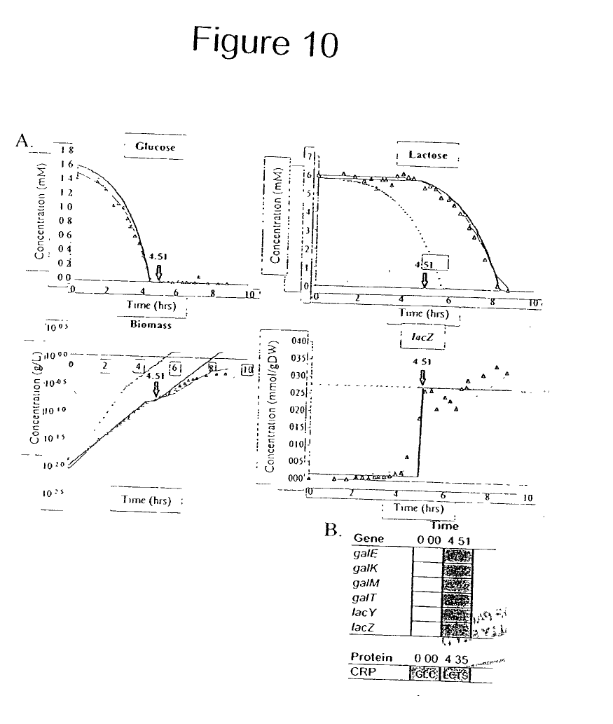

Aerobic growth of E. coli on glucose and lactose was simulated using the in silico models and compared to in vivo observations from mixed batch cultures and to results reported for a kinetic model as described in Kremling et al., Metabolic Ens. 3:362-379 (2001). Overall, the combined regulatory/metabolic model predictions were in good agreement with the in vivo observations, comparable with the predictions made by the Kremling model, and better than the predictions of the stand-alone metabolic model as shown in FIG. 10. The deficiencies in the ability of the stand-alone metabolic model to accurately predict the results of this experiment is most likely due to the concurrent uptake of glucose and lactose, resulting in much more rapid depletion of the substrates and a higher growth rate. Interestingly, because of the larger flux of carbon source uptake, the stand-alone metabolic model predicted that E. coli growth should be oxygen-, rather than carbon-limited in this case. Accordingly, the secretion of acetate and formate was predicted by the stand-alone metabolic model. In contrast, the combined regulatory/metabolic model predicted that no secretion will occur under these conditions.

The in silico arrays for the simulation (FIG. 10C) showed one shift in gene expression, occurring just under five hours. The up-regulation of the lactose uptake and degradation machinery, together with key enzymes in galactose metabolism, enables the system to use lactose as a carbon source once the glucose in the medium has been depleted.

The addition of regulatory constraints was used to interpret simulation results of cellular growth and by-product secretion. The glucose/acetate simulation indicated that upregulation of the glyoxalate shunt enables the reutilization of acetate, and that a second regulatory shift is responsible for regulation of genes such as ppsA and adhE, both of which were found to be regulated with no apparent reasons by unknown mechanisms in a recent microarray study of these conditions (Oh and Liao, Biotech. Progress 16:278-286 (2000)). The simulation of glucose-lactose diauxic growth indicated that upregulation of the gal and lac operons was vital to the diauxic shift observed.

By comparing the combined regulatory/metabolic simulations with those produced by the stand-alone metabolic model, it was possible to infer causes of regulatory evolution. In the case of glucose fermentation, the relatively small effect of regulation on the observed phenotype suggested that the organism has evolved a system which can respond instantaneously to sudden oxygen deprivation. Additionally, for the case of glucose-lactose diauxic growth, the stand-alone model showed that combined uptake of lactose and glucose could cause the system to be oxygen-, rather than carbon-limited for biomass production, resulting in the secretion of acetate and formate and reducing the growth yield. This finding, combined with evidence that E. coli evolves to optimize its growth yield during growth on single-carbon source media (Edwards et al., Nature Biotech. 1:125-130 (2001) and Ibarra et al., submitted) and that catabolite repression does not occur under starvation conditions, where the cell is carbon, rather than oxygen-limited (Lendenmann and Egli Microbiology 141:71-78 (1995)), suggests the hypothesis that regulation of substrate uptake may have evolved as a means of maintaining optimal growth yields on single substrates. Thus, the in silico models can be used to formulate hypotheses which address broad and fundamental topics such as regulatory network strategy.

These results demonstrate that the addition of regulatory constraints to a metabolic model can have a substantial impact on the simulation results, causing the simulation to better reflect the actual phenotype of a cell. These results further demonstrate that the combined regulatory/metabolic model has the ability to accurately capture behavioral features and systemic characteristics of central metabolism and regulation of E. coli with relatively few parameters.

Throughout this application various publications have been referenced. The disclosures of these publications in their entireties are hereby incorporated by reference in this application in order to more fully describe the state of the art to which this invention pertains.

Although the invention has been described with reference to the examples provided above, it should be understood that various modifications can be made without departing from the spirit of the invention. Accordingly, the invention is limited only by the claims.

TABLE 1

| Environments | | | | | Repressed Enzymes | | | | | | | Pathways |
|---|---|---|---|---|---|---|---|---|---|---|---|---|
| C1 | C2 | F | H | O2 | R2a | | R5b | R7 | R8a | | Tc2 | 26 |
| C1 | C2 | F | H | | R2a | R5a | | R7 | R8a | Rres | Tc2 | 10 |
| C1 | C2 | F | | O2 | | | R5b | | | | Tc2 | 8 |
| C1 | C2 | F | | | | R5a | | | | Rres | Tc2 | 4 |
| C1 | C2 | | H | O2 | R2a | | R5b | R7 | R8a | | Tc2 | 14 |
| C1 | C2 | | H | | R2a | R5a | | R7 | R8a | Rres | Tc2 | 5 |
| C1 | C2 | | | O2 | | | R5b | | | | Tc2 | 4 |
| C1 | C2 | | | | | R5a | | | | Rres | Tc2 | 2 |
| C1 | | F | H | O2 | R2a | | R5b | R7 | R8a | | Tc2 | 26 |
| C1 | | F | H | | R2a | R5a | | R7 | R8a | Rres | Tc2 | 10 |
| C1 | | F | | O2 | | | R5b | | | | Tc2 | 8 |
| C1 | | F | | | | R5a | | | | Rres | Tc2 | 4 |
| C1 | | | H | O2 | R2a | | R5b | R7 | R8a | | Tc2 | 14 |
| C1 | | | H | | R2a | R5a | | R7 | R8a | Rres | Tc2 | 5 |
| C1 | | | | O2 | | | R5b | | | | Tc2 | 4 |
| C1 | | | | | | R5a | | | | Rres | Tc2 | 2 |
| | C2 | F | H | O2 | R2a | | R5b | R7 | R8a | | | 26 |
| | C2 | F | H | | R2a | R5a | | R7 | R8a | Rres | | 10 |
| | C2 | F | | O2 | | | R5b | | | | | 8 |
| | C2 | F | | | | R5a | | | | Rres | | 4 |
| | C2 | | H | O2 | R2a | | R5b | R7 | R8a | | | 14 |
| | C2 | | H | | R2a | R5a | | R7 | R8a | Rres | | 5 |
| | C2 | | | O2 | | | R5b | | | | | 4 |
| | C2 | | | | | R5a | | | | Rres | | 2 |
| | | F | H | O2 | R2a | | R5b | R7 | R8a | | | 5 |
| | | F | H | | R2a | R5a | | R7 | R8a | Rres | | 0 |
| | | F | | O2 | | | R5b | | | | | 0 |
| | | F | | | | R5a | | | | Rres | | 0 |
| | | | H | O2 | R2a | | R5b | R7 | R8a | | | 2 |
| | | | H | | R2a | R5a | | R7 | R8a | Rres | | 0 |
| | | | | O2 | | | R5b | | | | | 0 |
| | | | | | | | | | | | | 0 |

TABLE 2

| | | | | |
|---|---|---|---|---|
| Reaction | Protein | Gene | Reaction | Regulatory logic |

A. Metabolic Fluxes

| | | | | |
|---|---|---|---|---|
| ACEA | Isocitrate lyase | aceA | ICIT -> GLX + SUCC | IF not (IclR) |
| ACEB | Malate synthase A | aceB | ACCOA + GLX -> COA + MAL | IF not (ArcA or IclR) |
| ACEE | Pyruvate dehydrogenase | aceEF, lpdA | PYR + COA + NAD -> NADH + CO2 + ACCOA | IF (not PdhR) |
| ACKAR | Acetate kinase A | ackA | ACTP + ADP <-> ATP + AC | |
| ACNAR | Aconitase A | acnA | CIT <-> ICIT | IF (GLCxt or LCTSxt or RIBxt or GLxt or LACxt or |
| | | | | PYRxt or SUCCxt or ETHxt or ACxt or FORxt) |
| ACNBR | Aconitase B | acnB | CIT <-> ICIT | IF (GLCxt or LCTSxt OR RIBxt or GLxt or LACxt or |
| | | | | PYRxt or SUCCxt or ETHxt or ACxt or FORxt) |
| ACS | Acetyl-CoA synthetase | acs | ATP + AC + COA -> AMP + PPI + ACCOA | IF not (GLCxt or LCTSxt or RIBxt or GLxt or LACxt or |
| | | | | PYRxt or SUCxt or ETHxt) and (not IClR) |
| ADHER | Acetaldehyde dehydrogenase | adhE | ACCOA + 2 NADH <-> ETH + 2 NAD + COA | IF not (O2xt) or not (O2xt and Cra) |
| ADK | Adenylate kinase | adk | ATP + AMP <-> 2 ADP | |
| ATPAR | FOF1-ATPase | atpABCDEFGHI | ATP <-> ADP P1 + 4 HEXT | |
| CYDA | Cytochrome oxidase bd | cydAB | QH2 + 5 O2 -> Q + 2 HEXT | IF (not FNR) or ArcA |
| CYOA | Cytochrome oxidase bo3 | cypABCD | QH2 + 5 O2 -> Q + 25 HEXT | IF not (ArcA or FNR) |
| DLD1R | D-Lactate dehydrogenase 1 | dld | PYR + NADH <-> NAD + LAC | |
| DLD2 | D-Lactate dehydrogenase (cytochrome) | dld | LAC + Q -> PYR + QH2 | |
| ENOR | Enotase | eno | 2PG <-> PEP | |
| FBAR | Fructose-1,6-bisphosphatate aldolase | fba | FDP <-> T3P1 + T3P2 | |
| FBP | Fructose-1,6-bisphosphatase | fbp | FDP -> F6P + P1 | |
| FDNG | Formate dehydrogenase-N | fdnGHI | FQR + Q -> QH2 + CO2 + 2 HEXT | IF FNR |
| FDOH | Formate dehydrogenase-O | fdoIHG | FQR + Q -> QH2 + CO2 + 2 HEXT | |
| FRDA | Fumarate reductase | frdABCD | FUM + FADH -> SUCC + FAD | IF FNR or DcuR |
| FUMAR | Fumarase A | fumA | FUM <-> MAL | IF not (ArcA OR FNR) |
| FUMBR | Fumarase B | fumB | FUM <-> MAL | IF FNR |
| FUMCR | Fumarase C | fumC | FUM <-> MAL | IF (superoxide radicals) |
| GALER | UDP-glucose 4-epimerase | galE | UDPGAL <-> UDPG | IF not (GLCxt) and not (GalR or GalS) |
| GALKR | Galactokinase | galK | GLAC + ATP <-> GAL1P + ADP | IF not (GLCxt) and not (GalR or GalS) |
| GALM1R | Aldose 1-epimerase (mutorotase) | galM | bDGLAC <-> GLAC | IF not (GLCxt) and not (GalR or GalS) |
| GALM2R | Aldose 1-epimerase (mutorotase) | galM | bDGLC <-> GLC | IF not (GLCxt) and not (GalR or GalS) |
| GALTR | Galactose-1-phosphate uridylyltransferase | galT | GAL1P + UTP <-> PPI + UDPGAL | IF not (GLCxt) and not (GalR or GalS) |
| GALUR | UDP-glucose-1-phosphate uridylyltransferase | galU | G1P + UTP <-> UDPG + PPI | |
| GAPAR | Glyceraldehyde-3-phosphate dehydrogenase-A complex | gapA | T3P1 + PI + NAD <-> NADH + 13PDG | |
| GLK | Glucokinase | glk | GLC + ATP + G6P + ADP | |
| GLPA | Glycerol-3-phosphate dehydrogenase (anaerobic) | glpABC | GL3P + Q -> T3P2 + QH2 | IF not (GLCxt or LCTSxt or RIBxt) and FNR and not |
| | | | | GlpR |
| GLPD | Glycerol-3-phosphate dehydrogenase (aerobic) | glpD | GL3P + Q -> T3P2 + QHZ | IF not (GLCxt or LCTSxt or RIBxt) and not (ArcA or |
| | | | | GlpR) |
| GLPK | Glycerol kinase | glpK | GL + ATP -> GL3P + ADP | IF not (GLCxt or LCTSxt or RIBxt) and not GlpR |
| GLTA | Citrate synthase | gltA | ACCOA + QA -> COA + CIT | |
| GND | 6-Phosphogluconate dehydrogenase (decarboxylating) | gnd | D6PGC + NADP -> NADPH + CO2 + RL5P | |
| GPMAR | Phosphoglycerate mutase 1 | gpmA | 3PG <-> 2PG | |
| GPMBR | Phosphoglycerate mutase 2 | gpmB | 3PG <-> 2PG | |
| GPSAR | Glycerol-3-phosphate-dehydrogenase-[NAD(P)+] | gpsA | GL3P + NADP <-> T3P2 + NADPH | |
| ICDAR | Isocitrate dehydrogenase | icdA | ICIT + NADP <-> CO2 + NADPH + AKG | |
| LACZ | Beta-galactosidase (LACTase) | lacZ | LCTS -> GLC + bDGLAC | IF not (GLCxt) and not (lacI) |
| MAEB | Malic enzyme (NADP) | maeB | MAL + NADP -> CO2 + NADPH + PYR | |
| MDHR | Malate dehydrogenase | mdh | MAL + NAD <-> NADH + OA | IF not ArcA |
| NDH | NADH dehydrogenase II | ndh | NADH + Q -> NAD + QH2 | IF not FNR |
| NUOA | NADH dehydrogenase I | nuoABEFGHIJKLMN | NADH + Q -> NAD + QH2 + 35 HEXT | |
| PCKA | Phosphoenolpyruvate carboxykinase | pckA | OT + ATP -> PEP + CO2 + ADP | |
| PFKA | Phosphofructokinase | pfkA | F6P + ATP -> FDP + ADP | |
| PFKB | Phosphofructokinase B | pfkB | F6P + ATP -> FDP + ADP | |
| PFLA | Pyruvate formate lyase 1 | pflAB | PYR + COA -> ACCOA + FOR | IF (ArcA or FNR) |
| PFLC | Pyruvate formate lyase 2 | pflCD | PYR + COA -> ACCOA + FOR | IF (Arca or FNR) |
| PGIR | Phosphoglucose isomerase | pgi | G6P <-> F6P | |
| PGKR | Phosphoglycerate kinase | pgk | 13PDG + ADP <-> 3PG + ATP | IF (GLCxt or LCTSxt or RIBxt or GLxt or LACxt or |
| | | | | PYRxt or SUCCxt or ETHxt or ACxt or FORxt) |
| PGL | 6-Phosphogluconolactonase | pgf | D6PGL -> D6PGC | |
| PGMR | Phosphoglucomutase | pgm | G1P <-> G6P | |
| PNTA1 | Pyridine nucleotide transhydrogenase | pntAB | NADPH + NAD -> NADP + NADH | |
| PNTA2 | Pyridine nucleotide transhydrogenase | pntAB | NADP + NADH + 2 HEXT -> NADPH + NAD | |
| PPA | Inorganic pyrophosphatase | ppa | PPI -> 2 PI | |
| PPC | Phosphoenolpyruvate carboxylase | ppc | PEP + CO2 -> OA + PI | |
| PPSA | Phosphoenolpyruvate synthase | ppsA | PYR + ATP -> PEP + AMP + PI | IF (Cra) |
| PTAR | Phosphotransacetylase | pta | ACCOA + PI <-> ACTP + COA | |
| PYKA | Pyruvate Kinase II | pykA | PEP + ADP -> PYR + ATP | |
| PYKF | Pyruvate Kinase I | pykF | PEP + ADP -> PYR + ATP | IF (not Cra) |
| RBSK | Ribokinase | rbsK | RIB + ATP -> R5P + ADP | IF not (GLCxt or LCTSxt) and not (RbsR) |
| RPER | Ribulose phosphate 3-epimerase | rpe | RL5P <-> X5P | |
| RPIAR | Ribose-5-phosphate isomerase A | rpiA | RL5P <-> R5P | |
| RPIBR | Ribose-5-phosphate isomerase B | rpiB | RL5P <-> R5P | IF not RpiR |
| SDHA1 | Succinate dehydrogenase | sdhABCD | SUCC + FAD -> FADH + FUM | IF not (ArcA or FNR) |
| SDHA2 | Succinate dehydrogenase complex | sdhABCD | FADH + Q <-> FAD + QH2 | IF not (ArcA OR FNR) |
| SFCA | Malic enzyme (NAD) | sfcA | MAL + NAD -> CO2 + NADH + PYR | |
| SUCA | 2-Ketoglutarate dehydrogenase | sucAB, lpdA | AKG + NAD + COA -> CO2 + NADH + SUCCOA | IF (not PdhR) |
| SUCCR | Succinyl-CoA synthetase | sucCD | SUCCOA + ADP + PI <-> ATP + COA + SUCC | |
| TALAR | Transaldolase A | talA | T3P1 + S7P <-> E4P + F6P | |
| TALBR | Transaldolase B | talB | T3P1 + S7P <-> E4P + F6P | |
| TKTA1R | Transketolase I | tktA | R5P + X5P <-> T3P1 + S7P | |
| TKTA2R | Transketolase I | tktA | X5P + E4P <-> F6P + T3P1 | |
| TKTB1R | Transketolase II | tktB | R5P + X5P <-> T3P1 + S7P | |
| TKTB2R | Transketolase II | tktB | X5P + E4P <-> F6P + T3P1 | |
| TPIAR | Triophosphate isomerase | tpiA | T3P1 <-> T3P2 | |
| ZWFR | Glucose 6-phosphate-1-dehydrogenase | zwf | G6P + NADP <-> D6PGL + NADPH | |

B. Transport Fluxes

| | | | | |
|---|---|---|---|---|
| ACUPR | Acetate transport | | ACxt + HEXT <-> AC | |
| CO2TXR | Carbon dioxide transport | | CO2xt <-> CO2 | |
| ETHUPR | Ethanol transport | | ETHxt + HEXT <-> ETH | |
| FORUPR | Formate transport | focA | FORxt <-> FOR | IF (ArcA or FNR) |
| | | | | IF (GLCxt or LCTSxt or RIBxt or GLxt or LACxt or |
| | | | | PYRxt or SUCCxt or ETHxt or ACxt or FORxt) and not |
| GLCPTS | Glucose transport | ptsGHI, crr | GLCxt + PEP -> G6P + PYR | Mlc) or (GLCxt or LCTSxt or RIBxt or GLxt or LACxt or |
| | | | | PYRxt or SUCCxt or ETHxt or ACxt or FORxt) and not |
| | | | | Cra) |
| GLCUP | Glucose transport (low affinity) | galP, etc. | GLCxt + HEXT -> GLC | IF (GLCxt or LCTSxt or RIBxt or GLxt or LACxt or |
| | | | | PYRxt or SUCCxt or ETHxt or ACxt or FORxt) |
| GLUPR | Glycerol transporter | glpF | GLxt <-> GL | IF not (GLCxt or LCTSxt or RIBxt) and not GlpR |
| LACUP | Lactate uptake | | LACxt + HEXT -> LAC | IF not (GLCxt or LCTsxt or RIBxt or GLxt) |
| LACDN | Lactate secretion | | LAC -> LACxt + HEXT | |
| LACYR | Lactose permease | lacY | LCTSxt + HEXT <-> LCTS | IF not (GLCxt) and not (lacI) |
| O2TXR | Oxygen transport | | O2xt <-> O2 | |
| PIUP2R | Phosphate transport | pitAB | PIxt + HEXT <-> PI | |
| PYRUPR | Pyruvate transport | | PYRxt + HEXT <-> PYR | |
| RIBUPR | Ribose transport | rbsABCD | RIBxt + ATP -> RIB +ADP + PI | IF not (GLCxt or LCTSxt) and not RbsR |
| DCTAR | Succinate transport | dctA | SUCCxt + HEXT <-> SUCC | IF not (GLCxt or LCTSxt or RIBxt or GLxt or LACxt or |
| | | | | PYRxt) and not ArcA and DcuR |
| DCUAR | Succinate transport | dcuA | SUCCxt + HEXT <-> SUCC | |
| DCUBR | Succinate transport | dcuB | SUCCxt + HEXT <-> SUCC | IF not (GLCxt or LCTSxt or RIBxt or GLxt or LACxt or |
| | | | | PYRxt) and FNR and DcuR |
| DCUC | Succinate efflux | dcuC | SUCC -> SUCCxt + HEXT | IF (FNR or ArcA) |

C. Maintenance/Biomass Production Fluxes

| | | |
|---|---|---|
| ATPM | ATP non-growth associated maintenance flux | ATP -> ADP + PI |
| | | 41.25 ATP + 3.54 NAD + 18.22 NADPH + 0.2 G6P + |
| | | 0.07 F6P + 0.89 R5P + 0.36 E4P + 0.12 T3P1 + 1.49 |
| VGRO | Biomass production flux | 3PG + 0.51 PEP + 2.83PYR + 3.74 ACCOA + 1.78 |
| | | OA + 1.07 AKG -> 3.74 COA + 41.25 ADP + 41.25 PI + |
| | | 3.54 NADH + 18.22 NADP + 1 Biomass |

D. Exchange Fluxes

| | | |
|---|---|---|
| ACex | Acetate exchange | ACxt -> |
| CO2ex | Carbon dioxide exchange | CO2xt -> |
| ETHex | Ethanol exchange | ETHxt -> |
| FORex | Formate exchange | FORxt -> |
| GLCex | Glucose exchange | GLCxt -> |
| GLex | Glycerol exchange | GLxt -> |
| Growth | Biomass exchange (growth) | Biomass + 13 ATP -> 13 ADP + 13 PI |
| LACex | Lactate exchange | LACxt -> |
| LCTSex | Lactose exchange | LCTSxt -> |
| O2ex | Oxygen exchange | O2xt -> |
| Plex | Phosphate exchange | Plxt -> |
| PYRex | Pyruvate exchange | PYRxt -> |
| RIBex | Ribose exchange | RIBxt -> |
| SUCCex | Succinate exchange | SUCCxt -> |

E. Regulatory Proteins

| | | |
|---|---|---|
| Aerobic/Anaerobic response regulator | acrA | active IF not (O2xt) |
| Cataborlite activator protein | cra (fruR) | active IF not (surplus FDP or F6P)* |
| Cataborlite repressor protein | crp | action is complex and highlighted in italics above |
| Dicarboxylate response regulator | dcuR | active IF DcuS |
| Dicarboxylate response sensor | dcuS | active IF (SUCCxt) |
| Fatty acid/Acetate response regulator | fadR | active IF (GLCxt) or not (ACxt) |
| Aerobic/Anaerobic response regulator | fnr | active IF not (O2xt) |
| Galactose operon repressor | galR | active IF not (GLAC) |
| Galactose operon repressor | galS | active IF not (GLAC) |
| Glycerol response regulator | glpR | active IF not (GLxt) |
| Fatty acid/Acetate response regulator | iclR | active IF FadR |
| Lactose operon repressor | lacI | active IF not (LCTSxt) |
| Glucose response regulator | mlc | active IF not (GLCxt) |
| Pyruvate response regulator | pdhR | active IF not (surplus PYR)** |
| Ribose response regulator | rbsR | active IF not (RIBxt) |
| Ribose response regulator | rprR | active IF not (RIBxt) |

TABLE 3

| | | | | | | glc | Dual |
|---|---|---|---|---|---|---|---|
| | glc | gl | suc | ac | rib | (—O₂) | Substrates |
| aceA | +/+/+ | | +/+/+ | −/−/− | | +/+/+ | |
| aceB | | | | −/−/− | | | |
| aceEF | −/+/− | | −/+/− | +/+/+ | | +/+/+ | (glc-ac) |
| | | | | | | | +/+/+ |
| ackA | | | | +/+/+ | | | |
| ackA + | | | | −/−/− | | | |
| pta + | | | | | | | |
| acs | | | | | | | |
| acnA | +/+/+ | +/+/+ | +/+/+ | +/+/+ | | +/+/+ | |
| acnB | +/+/+ | +/+/+ | +/+/+ | −/+/+ | | +/+/+ | |
| acnA + | −/−/− | −/−/− | −/−/− | −/−/− | | −/−/− | |
| acnB | | | | | | | |
| acs | | | | +/+/+ | | | |
| adh | +/+/+ | | | | | −/+/+ | |
| cyd | +/+/+ | | | | | | |
| cyo | +/+/+ | | | | | | |
| eno | −/−/− | −/−/− | −/−/− | | | | (gl-suc) |
| | | | | | | | +/+/+ |
| fbaA | −/+/+ | | | | | | |
| fbp | +/+/+ | −/−/− | −/−/− | −/−/− | | | |
| frdA | +/+/+ | | +/+/+ | +/+/+ | | +/+/+ | |
| fumA | | | | −/+/− | | +/+/+ | |
| gap | −/−/− | −/−/− | −/−/− | | | | (gl-suc) |
| | | | | | | | +/+/+ |
| glk | +/+/+ | | | | | | |
| glk + | +/+/+ | | | | | | |
| pfkA | | | | | | | |
| glk + | −/−/− | | | | | | |
| pts | | | | | | | |
| gltA | −/−/− | | | −/−/− | | | |
| gnd | +/+/+ | | | | | | |
| icd | −/−/− | | | −/−/− | | | |
| (idh) | | | | | | | |
| mdh | +/+/+ | +/+/+ | +/+/+ | | | +/+/+ | |
| ndh | +/+/+ | +/+/+ | | | | | |
| nuo | +/+/+ | +/+/+ | | | | | |
| pfl | | | | | | +/+/+ | |
| pgi | +/+/+ | +/−/− | +/−/− | | | | |
| pgi + | −/−/− | | | | | | |
| gnd | | | | | | | |
| pgi + | −/−/− | | | | | | |
| zwf | | | | | | | |
| pgk | −/−/− | −/−/− | −/−/− | | | | (gl-suc) |
| | | | | | | | +/+/+ |
| pgl | +/+/+ | | | | | | |
| ppc | −/+/− | −/+/− | +/+/+ | | | | (gl-suc) |
| | | | | | | | +/+/+ |
| | | | | | | | (glc-suc) |
| | | | | | | | +/+/+ |
| pta | | | | +/+/+ | | | |
| pts | +/+/+ | | | | | | |
| pykA | +/+/+ | | | | | | |
| pykA + | +/+/+ | | | | | | |
| pykF | | | | | | | |
| pykF | +/+/+ | | | | | | |
| rpiA | −/+/− | | | | +/+/+ | | (glc-rib) |
| | | | | | | | +/+/+ |
| rpiA + | −/−/− | | | | −/+/+ | | (glc-rib) |
| rbiB | | | | | | | +/+/+ |
| rpiB | +/+/+ | | | | +/+/+ | | (glc-rib) |
| | | | | | | | +/+/+ |
| rpiR + | +/N/+ | | | | +/N/+ | | (glc-rib) |
| rpiA | | | | | | | +/N/+ |
| sdhABCD | +/+/+ | | −/−/− | −/−/− | | +/+/+ | |
| sucAB- | −/+/+ | | −/+/+ | −/+/+ | | +/+/+ | (glc-suc) |
| lpd | | | | | | | +/+/+ |
| tpi | −/+/+ | −/−/− | −/−/− | −/−/− | | | (glc-suc) |
| | | | | | | | +/+/+ |
| | | | | | | | (glc-gl) |
| | | | | | | | +/+/+ |
| zwf | +/+/+ | | | | | | |

What is claimed is:

1. A computer readable medium or media, comprising:
(a) a data structure relating a plurality of reactants to a plurality of reactions of a biochemical reaction network,
wherein each of said reactions comprises a reactant identified as a substrate of the reaction, a reactant identified as a product of the reaction and a stoichiometric coefficient relating said substrate and said product, and wherein at least one of said reactions is a regulated reaction; and
(b) a constraint set for said plurality of reactions,
wherein said constraint set comprises a variable constraint for said regulated reaction.

2. The computer readable medium or media of claim 1, wherein said variable constraint is dependent upon the outcome of at least one reaction in said data structure.

3. The computer readable medium or media of claim 1, wherein said variable constraint is dependent upon the outcome of a regulatory event.

4. The computer readable medium or media of claim 1, wherein said variable constraint is dependent upon time.

5. The computer readable medium or media of claim 1, wherein said variable constraint is dependent upon the presence of a biochemical reaction network participant.

6. The computer readable medium or media of claim 5, wherein said participant is selected from the group consisting of a substrate, product, reaction., protein, macromolecule, enzyme and gene.

7. The computer readable medium or media of claim 1, wherein said biochemical reaction network comprises metabolic reactions.

8. The computer readable medium or media of claim 1, further comprising a regulatory data structure, wherein said variable constraint is dependent upon an outcome of a regulatory event represented by said regulatory data structure.

9. The computer readable medium or media of claim 8, wherein said regulatory data structure represents a regulatory event selected from the group consisting of transcription of a gene, translation of an RNA, post-translational modification of a protein, inhibition of a protein, activation of a protein, assembly of a protein, change in pH, change in redox potential, change in temperature, passage of time, and degradation of a protein.

10. The computer readable medium or media of claim 8, wherein said regulatory event is due to a signal transduction pathway.

11. The computer readable medium or media of claim 8, wherein said biochemical reaction network and said regulatory data structure represent reactions or events that occur in a single cell.

12. The computer readable medium or media of claim 8, wherein said biochemical reaction network represents reactions that occur in a first cell in a population of cells and said regulatory data structure represents events that occur in a second cell in said population.

13. The computer readable medium or media of claim 12, wherein said population of cells comprises cells of a multicellular organism.

14. The computer readable medium or media of claim 1, further comprising a constraint function that correlates an outcome of a regulatory event with said variable constraint.

15. The computer readable medium or media of claim 14, wherein said constraint function is binary.

16. The computer readable medium or media of claim 14, wherein said regulatory event is represented by Boolean logic.

17. The computer readable medium or media of claim 1, further comprising
(c) commands for determining at least one flux distribution that minimizes or maximizes an objective function when said constraint set is applied to said data structure, wherein said at least one flux distribution determines a systemic property of said biochemical reaction network, wherein said systemic property is dependent upon the flux through said regulated reaction.

18. The computer readable medium or media of claim 17, wherein said commands determine a range of feasible flux distributions that minimize or maximize an objective function when said constraint set is applied to said data representation.

19. The computer readable medium or media of claim 17, wherein said commands comprise an optimization problem.

20. The computer readable medium or media of claim 19, wherein said optimization problem comprises a linear optimization problem or a nonlinear optimization problem.

21. The computer readable medium or media of claim 17, further comprising a user interface capable of sending at least one command for modifying said data structure, said constraint set or said commands for applying said constraint set to said data representation, or a combination thereof.

22. The computer readable medium or media of claim 21, wherein said user interface further comprises links which a user may select to access additional information relating to said plurality of reactions.

23. The computer readable medium or media of claim 1, wherein said data structure comprises a set of linear algebraic equations.

24. The computer readable medium or media of claim 1, wherein said data structure comprises a matrix.

25. The computer readable medium or media of claim 1, further comprising commands for representing said at least one flux distribution as a flux distribution map.

26. The computer readable medium or media of claim 1, wherein at least one reactant in said plurality of reactants or at least one reaction in said plurality of reactions is annotated.

27. The computer readable medium or media of claim 26, wherein said annotation comprises assignment of said at least one reactant to a compartment.

28. The computer readable medium or media of claim 27, wherein a first substrate or product in said plurality of reactions is assigned to a first compartment and a second substrate or product in said plurality of reactions is assigned to a second compartment.

29. The computer readable medium or media of claim 26, wherein said annotation comprises assignment to an open reading frame or protein.

30. The computer readable medium or media of claim 26, wherein said annotation comprises a confidence rating.

31. The computer readable medium or media of claim 1, further comprising a gene database relating one or more reactions in said data structure with one or more genes or proteins in particular organism.

32. The computer readable medium or media of claim 1, wherein said biochemical reaction network comprises reactions that are selected from the group consisting of glycolysis, the TCA cycle, the pentose phosphate pathway, respiration, biosynthesis of an amino acid, degradation of an amino acid, biosynthesis of a purine, biosynthesis of a pyrimidine, biosynthesis of a lipid, metabolism of a fatty acid, biosynthesis of a cofactor, metabolism of a cell wall component, transport of a metabolite and metabolism of carbon, nitrogen, sulfur, phosphate, hydrogen or oxygen.

33. The computer readable medium or media of claim 1, wherein a plurality of said reactions are regulated reactions and said constraints for said regulated reactions comprise variable constraints.

34. A method for determining a systemic property of a biochemical reaction network, comprising:
(a) providing a data structure relating a plurality of reactants to a plurality of
reactions of a biochemical reaction network, wherein each of said reactions comprises a reactant identified as a substrate of the reaction, a reactant identified as a product of the reaction and a stoichiometric coefficient relating said substrate and said product, and wherein at least one of said reactions is a regulated reaction;
(b) providing a constraint set for said plurality of reactions,
wherein said constraint set comprises a variable constraint for said regulated reaction;
(c) providing a condition-dependent value to said variable constraint;
(d) providing an objective function, and
(e) determining at least one flux distribution that minimizes or maximizes said objective function when said constraint set is applied to said data structure,
thereby determining a systemic property of said biochemical reaction network.

35. The method of claim 34, wherein said value provided to said variable constraint changes in response to the outcome of at least one reaction in said data structure.

36. The method of claim 34, wherein said value provided to said variable constraint changes in response to the outcome of a regulatory event.

37. The method of claim 34, wherein said value provided to said variable constraint changes in response to time.

38. The method of claim 34, wherein said value provided to said variable constraint changes in response to the presence of a biochemical reaction network participant.

39. The method of claim 38, wherein said participant is selected from the group consisting of a substrate, product, reaction, enzyme, protein, macromolecule and gene.

40. The method of claim 34, wherein said biochemical reaction network comprises metabolic reactions.

41. The method of claim 34, further comprising a regulatory data structure, wherein said value provided to said variable constraint is changed due to an outcome of a regulatory event represented by said regulatory data structure.

42. The method of claim 41, wherein said regulatory event is selected from the group consisting of transcription of a gene, translation of an RNA, post-translational modification of a protein, inhibition of a protein, activation of a protein, assembly of a protein, change in pH, change in redox potential, change in temperature, passage of time, and degradation of a protein.

43. The method of claim 41, wherein said regulatory event is due to a signal transduction pathway.

44. The method of claim 41, wherein said biochemical reaction network and said regulatory data structure represent reactions or events that occur in a single cell.

45. The method of claim 41, wherein said regulatory event comprises a regulatory reaction.

46. The method of claim 41, wherein said biochemical reaction network represents reactions that occur in a first cell in a population of cells and said regulatory data structure represents events that occur in a second cell in said population.

47. The method of claim 46, wherein said population of cells comprises cells of a multicellular organism.

48. The method of claim 41, further comprising a constraint function that correlates an outcome of a regulatory event with said variable constraint.

49. The method of claim 48, wherein said constraint function is binary.

50. The method of claim 48, wherein said regulatory event is represented by Boolean logic.

51. The method of claim 48, wherein said constraint function correlates a first set of outcomes of said regulatory data structure with a first binary value and a second set of outcomes of said regulatory data structure with a second binary value.

52. The method of claim 48, wherein said constraint function correlates a set of outcomes of said regulatory data structure with a single integer value.

53. The method of claim 34, wherein said flux distribution is determined by optimization.

54. The method of claim 53, wherein said optimization comprises linear optimization or non linear optimization.

55. The method of claim 34, further comprising a step of modifying said data structure or said constraint set, or both.

56. The method of claim 34, wherein said data structure comprises a set of linear algebraic equations.

57. The method of claim 34, wherein said data structure comprises a matrix.

58. The method of claim 34, further comprising a step of producing a flux distribution map.

59. The method of claim 34, wherein said biochemical reaction network comprises reactions that are selected from the group consisting of glycolysis, the TCA cycle, pentose phosphate pathway, respiration, biosynthesis of an amino acid, degradation of an amino acid, biosynthesis of a purine, biosynthesis of a pyrimidine, biosynthesis of a lipid, metabolism of a fatty acid, biosynthesis of a cofactor, metabolism of a cell wall component, transport of a metabolite and metabolism of a carbon source, nitrogen source, oxygen source, phosphate source, hydrogen source or sulfur source.

60. The method of claim 34, wherein said systemic property is selected from the group consisting of growth, energy production, redox equivalent production, biomass production, production of biomass precursors, production of a protein, production of an amino acid, production of a purine, production of a pyrimidine, production of a lipid, production of a fatty acid, production of a cofactor, production of a cell wall component, transport of a metabolite, development, intercellular signaling, and consumption of carbon nitrogen, sulfur, phosphate, hydrogen or oxygen.

61. The method of claim 34, wherein said systemic property is selected from the group consisting of degradation of a protein, degradation of an amino acid, degradation of a purine, degradation of a pyrimidine, degradation of a lipid, degradation of a fatty acid, degradation of a cofactor and degradation of a cell wall component.

62. The method of claim 34, wherein said variable constraint comprises a condition-dependent constraint value and a constraint function, wherein said variable constraint is modified by said constraint function acting upon said condition-dependent constraint value.

63. The method of claim 62, wherein said constraint function is binary.

64. The method of claim 34, further comprising providing a gene database relating one or more reactions in said data structure with one or more open reading frames or proteins in a particular organism.

65. The method of claim 64, further comprising identifying an open reading frame that encodes a protein that performs a reaction in said plurality of reactions.

66. The method of claim 64, further comprising identifying a protein that performs a reaction in said plurality of reactions.

67. A method for determining a phenotype of a mutant of an organism, comprising:
(i) identifying a reaction that is not naturally present in a particular organism, and
(ii) determining a systemic property of a biochemical reaction network according to the method of claim 34, wherein said data structure relates a plurality of reactants for said organism to a plurality of reactions of a biochemical reaction network of said organism and further comprises said reaction that is not naturally present in said organism.

68. A method for determining a phenotype of a mutant of an organism, comprising:
(i) identifying a reaction that is related to an open reading frame or protein in said gene database, and
(ii) determining a systemic property of a biochemical reaction network according to the method of claim 34, wherein said reaction that is related to said open reading frame or protein is not present in said data structure or is constrained to have no flux.

69. A method for determining the effect of a drug on the activity of one or more reactions in a biochemical reaction network, comprising:
(i) identifying a reaction that is related to an open reading frame or protein in said gene database;
(ii) identifying a candidate drug that alters expression of said open reading frame or activity of said protein, and
(iii) determining a systemic property of a biochemical reaction network according to the method of claim 34, wherein said reaction that is related to said open reading frame or protein is not present in said data structure, is constrained to have a reduced flux, or is constrained to have no flux.

70. The method of claim 34, wherein a plurality of said reactions are regulated reactions and said constraints for said regulated reactions comprise variable boundary values.

71. A method for determining a systemic property of a biochemical reaction network at a first and second time, comprising:
(a) providing a data structure relating a plurality of reactants to a plurality of reactions of a biochemical reaction network,
wherein each of said reactions comprises a reactant identified as a substrate of the reaction, a reactant identified as a product of the reaction and a stoichiometric coefficient relating said substrate and said product, and wherein at least one of said reactions is a regulated reaction;
(b) providing a constraint set for said plurality of reactions, wherein said constraint set comprises a variable constraint for said regulated reaction;
(c) providing a condition-dependent value to said variable constraint;
(d) providing an objective function;
(e) determining at least one flux distribution at a first time that minimizes or maximizes said objective function when said constraint set is applied to said data structure,
thereby determining a systemic property of said biochemical reaction network at said first time;
(f) modifying said value provided to said variable constraint, and
(g) repeating step (e), thereby determining a systemic property of said biochemical reaction network at a second time.

72. The method of claim 71, wherein said value is modified based on said flux distribution at said first time.

73. The method of claim 71, wherein said value is modified based on a change in an environmental condition.

74. The method of claim 71, further comprising repeating steps (e) through (g) for a specified number of timepoints.
